(19) United States
(12) Patent Application Publication
(10) Pub. No.: US 2025/0012808 A1
(43) Pub. Date: Jan. 9, 2025

(54) PARALLEL ANTIBODY ENGINEERING COMPOSITIONS AND METHODS

(71) Applicants: The Broad Institute, Inc., Cambridge, MA (US); The General Hospital Corporation, Boston, MA (US)

(72) Inventors: Nir Hacohen, Cambridge, MA (US); Xun Chen, Cambridge, MA (US)

(73) Assignees: The Broad Institute, Inc., Cambridge, MA (US); The General Hospital Corporation, Boston, MA (US)

(21) Appl. No.: 18/750,005

(22) Filed: Jun. 21, 2024

Related U.S. Application Data
(63) Continuation of application No. PCT/US2022/082362, filed on Dec. 23, 2022.
(60) Provisional application No. 63/293,580, filed on Dec. 23, 2021.

(51) Int. Cl. G01N 33/68

(52) U.S. Cl. CPC G01N 33/6854

(57) ABSTRACT

The present invention discloses high-throughput methods for the creation of antibodies or antigen-binding fragments that can bind to single or multiple targets. Also disclosed are methods for using one or more antibodies or antigen-binding fragments to detect cognate binding partners in various types of samples.

### RELATED APPLICATIONS

The application claims the benefit under 35 U.S.C. 119 (e) of U.S. Provisional Application No. 63/293,580 filed Dec. 23, 2021, which is incorporated by reference in its entirety.

### BACKGROUND

The development of scalable, cost-effective antibody libraries for detecting proteins (or other molecules) has greatly lagged the development of methods for detecting specific RNA or DNA sequences, for example by hybridization or sequencing. Current applications that require antibody libraries rely on laborious and expensive barcoding of individual antibodies, followed by pooling for parallel detection. There remains a need for large-scale production of antibody libraries against multiple (e.g., hundreds to thousands) of human protein targets simultaneously

### SUMMARY

The present disclosure provides cell-free platforms that use distinct antibodies or antigen binding fragments (e.g., nanobodies with, for instance, >1011 complexity) to perform parallel selection for antibodies or antigen binding fragments that bind each of up to hundreds (to thousands) of cell surface proteins (e.g., a many-to-many screen). In some embodiments, the antibodies or antigen binding fragments are naturally barcoded with their encoding RNA by ribosome display, as disclosed in PCT/US2021/051925, which claims priority to U.S. Application Nos. 63/083,073 and 63/221,663, all three of which are incorporated by reference herein in their entirety.

The antibody or antigen binding fragment libraries disclosed herein can be engineered against diverse targets and large-scale libraries of barcoded antibodies that could be detected by nucleic acid hybridization or sequencing. These antibody or antigen binding fragment libraries could be used with many applications, including but not limited to high-dimensional cell cytometry (similar to CITE-seq, PMID: 28759029, PMID: 33649592), spatially indexed sequencing (e.g., using Slide-seq), in situ sequencing to detect antibodies bound to targets (similar to CODEX), and targeted proteomics (such as the Olink proximity extension assay).

Accordingly, some aspects of the disclosure provide a novel strategy to discover and engineer synthetic antibodies or antigen binding fragments, such as single variable domain on a heavy chain (VHH) nanobodies against hundreds to thousands of human protein targets simultaneously, which, according to some aspects provide a large collection of sequence-defined antibodies and enabling rapid synthesis of pools of barcoded-antibodies at large scale.

Also disclosed herein are embodiments wherein specific binders are identified by incubating antibodies or antigen binding fragments (e.g., RNA-barcoded nanobodies) with cells expressing a library of clustered regularly interspaced short palindromic repeats (CRISPR)/Cas9 single guide RNAs (sgRNAs) targeting genes, for instance, encoding proteins (e.g., cell surface proteins). Single-cell sequencing of antibody or antigen binding fragment (e.g., nanobody) sequences and sgRNA barcodes allows identification of sgRNAs associated with reduced binding of specific antibodies or antigen binding fragments (e.g., nanobodies), and thus map antibodies or antigen binding fragments (e.g., nanobodies) to proteins (e.g., cell surface proteins). The analysis of many targets at once systematically measures off-target binding for each antibody or antigen binding fragment (e.g., nanobody). The resulting library of surface-protein-binding antibodies or antigen binding fragments (e.g., nanobodies) enable single cell and spatial tissue proteomics. Building on the principle, the platform enables the development of detection (and perturbation) reagents against conventional and unconventional targets across many platforms.

In some embodiments, the methods for the identification of respective binding partners for a plurality of unique cognate antibodies or cognate antigen binding fragments thereof comprise:
(a) introducing into a population of cells a plurality of nucleic acids encoding a plurality of binding partners, which result in expression of a plurality of unique binding partners on the surface of the cells;
(b) incubating the cells obtained in step (a) with the plurality of unique antibodies or antigen binding fragments thereof under conditions that allow for binding to the plurality of unique binding partners; and
   wherein the plurality of unique cognate antibodies or cognate antigen binding fragments thereof is matched to their respective cognate binding partner if their binding to a cell in which the cognate binding partner is introduced is higher as compared to the binding to a cell in which the nucleic acid encoding the cognate binding partner was not expressed or expressed at a lower level.

In some embodiments, the cognate binding partner that binds its cognate antibody or cognate antigen binding fragment thereof is determined by compartmentalization of the nucleic acid encoding the cognate binding partners and the nucleic acid encoding the cognate antibody or cognate antigen binding fragment thereof. In other embodiments, the cognate binding partner that binds its cognate antibody or cognate antigen binding fragment thereof is determined by sequencing of the nucleic acid encoding the cognate binding partners and the nucleic acid encoding the cognate antibody or cognate antigen binding fragment thereof. In other embodiments, the sequencing is achieved by single-cell sequencing.

In some embodiments, the methods for the identification of respective binding partners for a plurality of unique cognate antibodies or cognate antigen binding fragments thereof comprise:
(a) providing a population of cells that express one or more cognate binding partners;
(b) disrupting and/or decreasing the expression of one or more cognate binding partners in one or more cells of the population; and
(c) incubating the population of cells in step (b) with a plurality of unique antibodies or antigen binding fragments thereof under conditions that allow for the binding of cognate antibodies or cognate antigen binding fragments thereof to their respective cognate binding partner;
(d) determining which cognate antibodies or cognate antigen binding fragments thereof did not bind or had decreased binding to one or more cells of the population with disrupted and/or decreased cognate binding partner expression,
   wherein the respective cognate binding partners for the plurality of cognate antibodies or cognate antigen binding fragments thereof is identified when the cognate antibodies or cognate antigen binding fragments thereof is determined to have not bound or had lower binding to the cell in which the cognate binding partner is disrupted and/or decreased.

In some embodiments, the expression of the one or more cognate binding partners is disrupted and/or decreased using a clustered regularly interspaced short palindromic repeats (CRISPR)-based system. In other embodiments, the one or more cells in which the expression of one or more cognate binding partners is disrupted and/or decreased also express a gene editing nuclease. In other embodiments, the gene editing nuclease is an endonuclease. In other embodiments, the gene editing nuclease is Cas9.

In some embodiments, the expression of one or more cognate binding partners is disrupted and/or decreased by contacting the population of cells with a ribonucleic acid that is a single-guide RNA (sgRNA). In other embodiments, the expression of the one or more cognate binding partners is disrupted and/or decreased using RNA interference (RNAi). In other embodiments, the expression of the one or more cognate binding partners is disrupted and/or decreased by contacting the population of cells cell with a ribonucleic acid which is either a small interfering RNA (siRNA) or a short hairpin RNA (shRNA), thereby introducing the ribonucleic acid into individual cells and knocking-down the expression of the cognate binding partner that is the target of the ribonucleic acid in the cells in which the ribonucleic acid was introduced. In other embodiments, the cognate antibodies or cognate antigen binding fragments thereof are linked to nucleic acids containing their respective coding sequences or fragment thereof. In other embodiments, the cognate antibodies or cognate antigen binding fragments thereof are linked to nucleic acids containing their respective coding sequences or fragment thereof using the ribonucleic acid. In other embodiments, the ribonucleic acid responsible for disrupting and/or decreasing the cognate binding partner and the nucleic acid encoding the cognate antibody or cognate antibody binding fragment thereof is determined by compartmentalization of the nucleic acid encoding the cognate binding partners and the nucleic acid encoding the cognate antibody or cognate antigen binding fragment thereof. In other embodiments, the ribonucleic acid responsible for disrupting and/or decreasing the cognate binding partner and the nucleic acid encoding the cognate antibody or cognate antibody binding fragment thereof is determined by sequencing of the nucleic acid encoding the cognate binding partners and the nucleic acid encoding the cognate antibody or cognate antigen binding fragment thereof. In other embodiments, the sequencing is achieved by single-cell sequencing.

In some embodiments, the ribonucleic acid responsible for disrupting and/or decreasing the cognate binding partner is used to identify the cognate binding partner with disrupted expression in the cell. In other embodiments, the population of cells is contacted with a plurality of sgRNAs or shRNAs at a multiplicity of infection (MOI) less than 1. In other embodiments, the MOI is 0.2, 0.3, 0.4, 0.5, 0.6, 0.7, 0.8, or 0.9. In other embodiments, the plurality of sgRNAs or shRNAs is introduced into the population of cells using lentiviral transduction. In other embodiments, the plurality of sgRNAs or shRNAs is introduced into the population of cells using transformation, transfection, or electroporation. In other embodiments, the plurality of sgRNAs or shRNAs comprises 2 to 100,000 different ribonucleic acids encoding different sgRNAs or shRNAs that target expression of different genes encoding different binding partners.

In some embodiments, the antibodies or antigen binding fragments thereof are associated with the nucleic acids encoding their respective coding sequences. In other embodiments, the association of the antibodies or antigen binding fragments thereof to the nucleic acids encoding their respective coding sequences is achieved using ribosome display complexes comprising the antibodies or antigen binding fragments thereof linked to the nucleic acids encoding their coding sequences and ribosomes. In other embodiments, the nucleic acids encoding the antibody or antigen binding fragment thereof coding sequence are RNA. In other embodiments, the ribosomes are from eukaryotic cells. In other embodiments, the ribosomes are from prokaryotic cells.

In some embodiments, the respective cognate binding partners are proteins. In other embodiments, the cognate antibody or cognate antigen binding fragment thereof that bind a cognate binding partner are clustered computationally. In other embodiments, a cluster size of a cognate antibody or cognate antigen binding fragment thereof cluster is assessed by counting the number of sequences in the cluster. In other embodiments, higher or lower binding of the cognate antibody or cognate antigen binding fragment thereof to a cognate binding partner is determined by a larger or smaller cluster size of the cluster representing the cognate antibody or cognate antigen binding fragment thereof. In other embodiments, the cluster is normalized relative to the total sequence number to obtain a relative cluster size.

In other embodiments, a specificity value is calculated for a cluster of cognate antibody or cognate antigen binding fragment by calculating the fraction of cluster members associated with each binding partners among a plurality of binding partners and finding the largest value. In other embodiments, an affinity value is calculated for a cluster of cognate antibody or cognate antigen binding fragment for a binding partner by calculating the count of cluster members associated with the binding partner. In other embodiments, the ranking of the performance of a plurality of cognate antibody or cognate antigen binding fragment is obtained by ranking their cluster size and/or specificity value and/or affinity value. In other embodiments, the performance metrics of a cognate antibody or cognate antigen binding fragment is obtained by obtaining their cluster size and/or specificity value and/or affinity value.

In some embodiments, the methods for obtaining a plurality of unique cognate antibodies or cognate antigen binding fragments thereof that bind to their respective cognate binding partner present on a surface comprising:
(a) incubating a plurality of unique antibodies or antigen binding fragments thereof with one or more binding partners bound to the surface under conditions that allow for binding of the antibodies or antigen binding fragments thereof to their respective cognate binding partner;
(b) collecting a plurality of antibodies or antigen binding fragments thereof that had bound to their respective cognate binding partner;

wherein the plurality of unique cognate antibodies or cognate antigen binding fragments thereof that bind to their respective cognate binding partner are obtained.

In some embodiments, the surface is the surface of one or more target cells. In other embodiments, the plurality of unique antibodies or antigen binding fragments thereof of step (a) is obtained by:
(aa) incubating a preliminary plurality of unique antibodies or antigen binding fragments thereof with a non-target surface under conditions that allow for the binding of antibodies or antigen binding fragments thereof to the non-target surface;
(bb) collecting the antibodies or antigen binding fragments thereof that did not bind to the non-target surface in step (aa), wherein the plurality of unique antibodies or antigen binding fragments thereof of step (a) is obtained.

In some embodiments, the non-target surface is the surface of one or more non-target cells. In other embodiments, the non-target cells do not express, or express at lower levels as compared to the one or more target cells of, the cognate binding partners. In other embodiments, the one or more target cells comprise cells in suspension or attached to a substrate. In other embodiments, the one or more target cells are fixed using a fixation reagent. In other embodiments, the fixation reagent is formaldehyde.

In some embodiments, the one or more target cells are one or more of a T cell population, a kidney cell population, or a bone marrow cell population. In other embodiments, the one or more target cells comprise blood cells. In other embodiments, the one or more target cells comprise cancer cells. In other embodiments, the one or more target cells comprise leukemia cells or neuroblastoma cells. In other embodiments the one or more target cells comprise leukemic T-cell lymphoblasts. In other embodiments, the one or more target cells comprise Jurkat cells, HEK-293 cells, SH-SY5Y cells, or CHO cells. In other embodiments, the one or more target cells are cultured for 1 day to 14 days prior to step (a).

In some embodiments, the non-target cells are phylogenetically distant from the one or more target cells. In other embodiments, the non-target cells and the one or more target cells are from different organs or different species. In other embodiments, the plurality of antibodies or antigen binding fragments thereof of step (b) comprises the steps of (1) incubating the surface in a buffer comprising chelating agents, (2) fractionating the incubated surface and buffer, thereby producing a supernatant that does not comprise the surface, and (3) retaining the supernatant after the fractionation of step (2). In other embodiments, the chelating agent is EDTA or EGTA. In other embodiments, the one or more target cells express one or more cancer driver genes and/or genes carrying cancer driver mutations, and the cognate binding partners are expressed by at least one of the one or more target cells and are induced by the cancer driver genes and/or genes carrying cancer driver mutations.

In some embodiments, the cognate binding partner proteins are natively expressed proteins. In other embodiments, the cognate binding partner proteins are expressed by the one or more target cells by introducing nucleic acids containing the coding nucleic acid sequences for the cognate binding partner proteins into the cells. In other embodiments, the respective cognate binding partner proteins have post-translational modifications. In other embodiments, the respective cognate binding partner protein is part of a protein complex. In other embodiments, the respective cognate binding partners comprise a lipid group or a sugar group. In other embodiments, the respective cognate binding partners are not proteins. In other embodiments, the respective cognate binding partners are lipids or sugars. In other embodiments, the respective cognate binding partners are from a mammal. In other embodiments, the respective cognate binding partners are from a mouse. In other embodiments, the respective cognate binding partners are from a human.

In some embodiments, the respective cognate binding partner is a cell surface protein. In other embodiments, the respective cognate binding partners are membrane proteins or membrane protein domains, intracellular proteins or intracellular protein domains, cytosolic proteins or cytosolic protein domains, or nuclear proteins or nuclear protein domains. In other embodiments, the respective cognate binding partners are peptides. In other embodiments, the peptides are 2 amino acids to 30 amino acids in length. In other embodiments, the peptides are part of a peptide-carrier protein fusion. In other embodiments, the respective cognate binding partner comprises a complex of a peptide loaded onto a major histocompatibility complex protein. In other embodiments, the respective cognate binding partners are proteins that mediate cell-to-cell interactions. In other embodiments, the respective cognate binding partners are proteins that are overexpressed in cancer cells or tumor cells. In other embodiments, the respective cognate binding partner is an ecto-nucleoside triphosphate diphosphohydrolase 1, E-NTPDasel (CD39), a T cell immunoglobulin and mucin domain-3 (Tim3), a cluster of differentiation 28 (CD28), a cluster of differentiation 4 (CD4), and/or a stimulator of interferon gene (STING).

In some embodiments, the cognate antibody or cognate antigen binding fragment thereof has an affinity for its respective cognate binding partner of a K_{D} of less than 10 μM. In other embodiments, the respective cognate binding partners are cancer drug targets or a cancer immunotherapy targets.

In some embodiments, the methods for the detection of respective cognate binding partners in a sample comprise:
(a) incubating a sample comprising a population of cells expressing cognate binding partners with a plurality of ribosome display complexes, wherein the ribosome display complexes comprise:
   (1) a plurality of unique antibodies or antigen binding fragments thereof,
   (2) nucleic acids encoding the plurality of unique antibodies or antigen binding fragments thereof, and
   (3) ribosomes, under conditions that allow for binding of the plurality of the antibodies or antigen binding fragments thereof to their respective cognate binding partners expressed by the cells of the sample;
(b) detecting the ribosome display complexes that bound to their cognate binding partners in the sample; and
(c) quantifying the cognate antibodies or cognate antigen binding fragments thereof bound to their cognate binding partners expressed by the cells of the sample, thereby detecting the respective cognate binding partners in the sample.

In some embodiments, at least one nucleic acid encoding an antibody or antigen binding fragment thereof comprises a unique barcode in the open reading frame (ORF). In other embodiments, the sample is selected from the group consisting of single cells, tissue slices, organs, and organisms. In other embodiments, In other embodiments, step (c) further comprises sequencing one or more of the barcodes. In other embodiments, the barcodes are the nucleic acid sequence of CDR1, CDR2, or CDR3 of the cognate antibodies or antigen binding fragments thereof. In other embodiments, step (b) is achieved by using secondary antibodies. In other embodiments, step (b) is achieved by fluorescence in situ hybridization.

In some embodiments, a plurality of nucleic acids encoding the antibodies or antigen binding fragments thereof is used to determine a surface proteome of a cell. In other embodiments, the surface is one or more target cells, and wherein a surface proteome of a cell is determined using flow cytometry, imaging, proteomic analysis, or functional analysis. In other embodiments, the surface proteome of a cell in the cell population is determined using flow cytometry, imaging, proteomic analysis, or functional analysis. In other embodiments, includes sequencing a plurality of nucleic acids encoding the antibodies or antigen binding fragments thereof, clustering the nucleic acid sequences computationally, synthesizing one or more sequences from one or more clusters of antibodies or antigen binding fragments thereof for cloning of the respective genes, and testing the respective antibodies or antigen binding fragments thereof.

In some embodiments, the cognate antibodies or cognate antigen binding fragments thereof are allowed to bind to their respective cognate binding partners in the presence of ribonuclease inhibitors. In other embodiments, the ribonuclease inhibitor is a protein or a small molecule. In other embodiments, the ribonuclease inhibitor is Ribonucleoside Vanadyl Complex.

In other embodiments, the antibodies or antigen binding fragments thereof are nanobodies. In other embodiments, the antibodies or antigen binding fragments thereof are a variable domain of the heavy chain (VHH). In other embodiments, the antibodies or antigen binding fragments thereof are modified to alter stability, aggregation propensity, in vivo half-life, neutralizing activity and/or dimerization. In other embodiments, the antibodies or antigen binding fragments thereof are fusion proteins. In other embodiments, the antibodies or antigen binding fragments thereof are fused to another antibody or antibody fragment thereof, Fc domain, antigen binding domain, glutathione S-transferase (GST), small molecule, and/or serum albumin. In other embodiments, the respective cognate binding partner is an antigen.

In some embodiments, the composition comprises a cognate antibody or cognate antigen binding fragment thereof associated with a respective cognate binding partner, a target cell, and a nucleic acid inside the target cell encoding, or interfering the expression of, the respective cognate binding partner. In other embodiments, the target cell is a T cell, a kidney cell, or a bone marrow cell. In other embodiments, the target cell is a blood cell. In other embodiments, the target cell is a cancer cell. In other embodiments, the target cell is a leukemia cell or a neuroblastoma cell. In other embodiments, the target cell is a leukemic T-cell lymphoblast. In other embodiments, the target cell is a Jurkat cell, a HEK-293, an SH-SY5Y cell, or a CHO cell.

In some embodiments, the respective cognate binding partner is a cancer drug target. In other embodiments, the respective cognate binding partner is a protein or a protein domain. In other embodiments, the protein or protein domain has a post-translational modification. In other embodiments, the respective cognate binding partner is part of a protein complex. In other embodiments, the respective cognate binding partner comprises a lipid group or a sugar group.

In some embodiments, the respective cognate binding partner is not a protein. In other embodiments, the respective cognate binding partner is a lipid. In other embodiments, the respective cognate binding partner is a sugar. In other embodiments, the respective cognate binding partner is from a mammal. In other embodiments, the respective cognate binding partner is from a mouse. In other embodiments, the respective cognate binding partner is from a human.

In some embodiments, the respective cognate binding partner comprises a cell surface protein. In other embodiments, the respective cognate binding partner comprises a membrane protein or membrane protein domain. In other embodiments, the respective cognate binding partner comprises an intracellular protein. In other embodiments, the respective cognate binding partner comprises is a cytosolic protein. In other embodiments, the respective cognate binding partner comprises a nuclear protein. In other embodiments, the respective cognate binding partner comprises a peptide. In other embodiments, the respective cognate binding partner comprises a complex of a peptide loaded onto a major histocompatibility complex protein.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1C are schematics of massively parallel nanobody generation and nanobody-target mapping by ORF expression. A three-stage process to generate nanobodies to multiple (one to thousands) selected protein domains (targets). FIG. 1A shows the targets expressed as artificial cell membrane proteins. FIG. 1B shows nanobodies against any of the selected targets are selected by pooled selection which yields an output DNA library encoding nanobodies that can bind one of the selected targets. FIG. 1C Nanobody-target mapping: the target of each nanobodies in the output DNA library is found by single cell sequencing.

FIGS. 2A-2C are schematics showing massively parallel nanobody generation and nanobody-target mapping by Knock-out-Drop-out. FIG. 2A shows a cell line is chosen that expresses native surface protein of interest (target cell). Another cell line of different origin is used for pre-clearing (a non-target cell). A lentivirus library is built that can knock out surface proteins on the target cells. FIG. 2B shows nanobodies against any of the surface proteins on the target cells are selected by pooled selection which yields an output DNA library encoding nanobodies that can bind one of the surface proteins. FIG. 2C Nanobody-target mapping: the target of each nanobodies in the output DNA library is found by single cell sequencing.

FIG. 3 is a schematic for sequencing the NGS sequencing library in nanobody-target mapping. Four reads are performed that covers CDR1, CDR2, CDR3 and the single cell barcode. The full-length sequence is obtained by filling in with the constant frame region sequences.

FIG. 4 is a schematic of using ribosome display complex pools as highly multiplex protein profiling reagents. A large library of nanobodies with known targets can be displayed by ribosome display. The resulting solution can be used to profile protein levels by quantifying nanobody RNA sequences/barcodes using high-throughput sequencing.

FIGS. 5A-5B is a schematic of using whole cells to present antigen on the cell surface for nanobody selection. FIG. 5A. Using EGFP as an example, a protein can be expressed as an artificial cell surface protein by adding a N-terminal signal peptide and a C-terminal GPI anchor sequence. FIG. 5B. Whole cells can act as solid surfaces for presenting antigen for selection of nanobody ribosome display complex, the receptor binding domain (RBD) of SARS-COV-2 spike protein and RBD nanobodies are used to test feasibility, EGFP is used as negative control.

FIGS. 6A-6F shows a method for cell-free nanobody engineering. FIG. 6A. is a schematic of the structure of the linear DNA library. FIG. 6B. is a schematic of in vitro translation of the DNA library yielding RNA-ribosome-nanobody complexes. FIG. 6C. is a schematic of a library of such complexes that is iteratively applied to immobilized targets on solid surfaces to enrich for binder sequences in the output library. FIG. 6D. is a schematic of full-length sequencing and computational clustering of the output library providing a comprehensive list of nanobody clusters; each cluster represent one nanobody. FIG. 6E. is a schematic that shows the selection against SARS-COV-2 RBD generates hundreds of nanobody clusters; and FIG. 6F. is a graph showing potent neutralizing nanobodies.

FIG. 7 is a schematic for an integrated VHH antibody discovery platform comprising four parts. FIG. 7 (top left) shows a synthetic universal VHH (also known as nanobody) library with randomized CDR regions, the library molecules are linear DNA and contain 5 prime and 3 prime sequence elements required for ribosome display. FIG. 7 (top right) shows genotype-phenotype complexes formed by ribosome display of the synthetic universal VHH library. FIG. 7 (bottom left) shows selection for binders to immobilized target antigen from the genotype-phenotype complexes, yielding an output library composed of DNA encoding VHH binders. FIG. 7 (bottom right) shows binder discovery from the selection output library using high-throughput sequencing and CDR-directed clustering.

FIG. 8 is a schematic for parallel antibody discovery approaches that use massively parallel antibody discovery achieved by pooled antibody selection followed by antibody-antigen mapping. FIG. 8 (top) shows pooled antibody selection performed using antibody selection against a pool of antigens to recover a pool of DNA molecules encoding binders. FIG. 8 (bottom) shows antibody-antigen mapping that maps antibodies recovered by pooled antibody selection to their cognate antigen in a high-throughput manner.

FIG. 9 shows quantitative detection of nucleic acid sequences of bound VHH on cells and antigens expressed by the cells. Equal amounts of ribosome display complexes of four VHHs were incubated with a mixture of cells with each cell expressing either the receptor binding domain of SARS-COV-2 spike (RBD) or EGFP on the cell surface. The cells with bound ribosome display complexes were then compartmentalized and sequenced by droplet-based single cell sequencing to quantify the unique molecular identifier (UMI) of VHH and antigen nucleic acid sequences. Compartments containing RBD expressing cells were enriched with anti-RBD VHHs (top), and compartments containing EGFP expressing cells (bottom) were enriched with anti-EGFP VHHs, while compartment containing both RBD and EGFP cells (middle) showed a more even mixture of anti-RBD and anti-EGFP VHHs.

FIGS. 10A-10B show antibody-antigen mapping using anti-RBD VHHs as positive controls. Equal amount of DNA encoding SR1 (FIG. 10A) or SR6v7 (FIG. 10B), two VHHs that bind RBD, were included as input for ribosome display, the formed ribosome display complexes were incubated with cells expressing different antigens (x axis) on the cell surface. The cells were then sequenced by droplet-based single cell sequencing to quantify the unique molecular identifier (UMI) of VHH sequences (count) and antigen sequences to determine antigen identity. The cell number normalized fraction of each VHH on cells expressing each antigen were calculated (y axis). The anti-RBD VHHs are enriched on cells expressing RBD. The count per RBD cell value of a VHH correlates with the VHH's affinity. The cell number normalized fraction of a VHH on cells expressing the VHH's cognate antigen correlates with the VHH's specificity.

FIG. 11 shows the mapping of VHHs recovered from a pooled selection against a pool of antigens to their cognate antigen. Pooled VHH selection was performed against a pool of antigens (y axis, except RBD which is not included during selection). The cell number normalized fraction of each VHH cluster (x axis) on cells expressing each antigen were calculated (marker size). The specificity values are the highest fraction among all antigens for each VHH cluster. As such, the specificity value describes the specificity of an antibody towards a collection of antigens. The specificity value is measured by incubating a defined amount of the antibody with the collection of antigens in defined amounts under a defined condition and finding the percentage of antibodies bound to each antigen, the highest percentage value among the values for each antigen is the specificity value. SR1 and SR6v7 are previously identified anti-RBD VHHs spiked in as positive controls, VHH1-22 are representative VHH clusters recovered by the pooled selection.

### DETAILED DESCRIPTION

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Embodiments disclosed herein provide antibody engineering platforms for rapid isolation of large-scale number of antibodies and antigen-binding fragments thereof in which antibody or antibody binding fragments thereof (such as nanobodies) are identified as matching one more cognate binding partners, and the antibodies or antibody binding fragments thereof obtained by the platform. These large-scale number of antibody libraries will be useful for, among other reasons, detecting the cell surface proteome for cytometry, imaging, proteomic or functional studies. The engineering platform described here also provides an approach to find binders of “difficult” targets (e.g., protein complexes, post-translational modifications, non-protein targets such as sugars), and methods for producing programmable pooled libraries of barcoded nanobodies. Antibodies are widely used in research and medicine. Conventional antibody generation by immunizing animals is time-intensive and leads to variable results. A cell-free antibody engineering platform (CeVICA) has previously been described that integrates a synthetic library of camelid heavy-chain antibody VHH domains with randomized complementarity-determining regions (CDRs), optimized in vitro selection based on ribosome display and a computational pipeline for binder prediction based on CDR-directed clustering. The present disclosure, among other things, provides for the high-throughput identification of large numbers of antibody or antigen-binding fragments thereof, for example nanobodies, that bind to specific antigens, (such as, but not necessarily limited to, proteins) in a cell, such as cell surface proteins, but also with randomized complementarity-determining regions (CDRs) and optimized in vitro selection based on ribosome display and a computational pipeline for binder prediction based on CDR-directed clustering.

### Antibodies

In some embodiments, the present invention provides antibodies, antibody fragments, binding fragments of an antibody, or antigen binding fragments thereof, capable of binding to an antigen of interest. The term “antibody” is used interchangeably with the term “immunoglobulin” herein, and includes intact antibodies, fragments of antibodies, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)₂, Fabc, Fd, dAb, VHH (nanobody), VH, VL, and scFv and/or Fv fragments.

The term “antigen binding fragment” refers to a polypeptide fragment of an immunoglobulin or antibody that binds antigen or competes with intact antibody (e.g., with the intact antibody from which they were derived) for antigen binding (e.g., specific binding). As such, these antibodies or fragments thereof are included in the scope of the invention, provided that the antibody or antigen binding fragment thereof (e.g., cognate antibody, cognate antigen binding fragment, nanobody, etc.) binds specifically to a binding partner (e.g., target molecule, cognate binding partner or respective cognate binding partner).

In some embodiments, the antibody, antibody fragment or antigen binding fragment is a therapeutic antibody or therapeutic antigen binding fragment thereof. In some embodiments, the antibody, antibody fragment or antigen binding fragment thereof is a neutralizing antibody, neutralizing antibody fragment or neutralizing antigen binding fragment.

As used herein, “neutralizing antibody” refers to an antibody, antibody fragment or antigen binding fragment thereof that is capable of neutralizing a pathogen or reducing infectivity (e.g., viral pathogen or bacterial pathogen).

As used herein “complementarity determining regions” or “CDRs” refer to variable regions in an antibody that provide for antigen specificity. In some embodiments, specific CDRs identified can be used in an antibody framework described further herein. In some embodiments, one, two, or all three CDRs are used in a framework. In some embodiments, CDR1 and CDR3 are used in a framework. In some embodiments, CDR3 is used in a framework. In preferred embodiments, all three CDRs are used in a framework. In some embodiments, the CDRs are used in a heavy-chain antibody VHH domain. As used herein, framework can refer to an entire antibody VHH domain or antibody as described herein. In some embodiments, frame region (FR) refers to the non-CDR regions or constant regions in the antibody. The frame regions in the antibodies of the present invention are also referred to as frame1, frame2, frame3 and frame4.

In some embodiments, the antibodies of the present invention are heavy chain antibodies. As used herein “heavy chain antibody,” “VHH” or “single-domain antibodies” (sdAbs) refers to an antibody which consists only of two heavy chains and lacks the two light chains usually found in antibodies (see, e.g., Henry and Mackenzie, Antigen recognition by single-domain antibodies: structural latitudes and constraints. MAbs. 2018 August-September; 10 (6): 815-826). VHH can refer to an antibody or VHH domain. Single-domain antibodies (sdAb), which can be known as nanobodies; an antibody fragment consisting of a single monomeric variable antibody domain. The ˜12-15 kDa variable domains of these antibodies (VHHs and VNARs) can be produced recombinantly and can recognize antigen in the absence of the remainder of the antibody heavy chain. In common antibodies, the antigen binding region consists of the variable domains of the heavy and light chains (VH and VL). Heavy-chain antibodies can bind antigens despite having only VH domains. In some embodiments, the heavy chain antibody is an antibody derived from cartilaginous fishes (immunoglobulin new antigen receptor (IgNAR)) or camelid ungulates. Non-limiting examples of camelids include dromedaries, camels, llamas, and alpacas.

In some embodiments, antibodies or antigen binding fragments thereof prepared according to the present invention are substantially free of non-antibody protein. As used herein, a preparation of antibody protein having less than about 50% of non-antibody protein (also referred to herein as a “contaminating protein”), or of chemical precursors, is considered to be “substantially free.” Substantially free is considered to be 40%, 30%, 20%, 10% and more preferably 5% (by dry weight), free of non-antibody protein, or of chemical precursors. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

In preferred embodiments, the antibodies of the present invention are monoclonal antibodies. As used herein, the term “monoclonal antibody” refers to a single antibody produced by any means, such as recombinant DNA technology. As used herein, the term “monoclonal antibody” also refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F (ab′)₂, Fabc, Fd, dAb, Fv, single chains, VHH, single-chain antibodies, e.g., scFv, and single domain antibodies.

In some embodiments, “humanized” forms of non-human antibodies contain amino acid residues in frame regions that resemble human antibody frame regions. In some embodiments, frame regions of camelid antibodies or heavy chain antibodies are modified. In some embodiments, humanized residues can be found in any human IGHV gene. In some embodiments, the humanized residues are located in frame 2 or frame 4. In preferred embodiments, the humanized residues are located in frame 2 position 4, frame 2 position 11, frame 2 position 12, frame 2 position 14, frame 4 position 8. Humanized frames can be based on well characterized VHHs (Kirchhofer et al., 2010; Turner et al., 2014). These frames share high homology with the human IGHV3-23 or IGHJ4, but can be altered further as described herein (e.g., Frames 2 and 4).

In some embodiments, “humanized” forms of non-human antibodies are chimeric antibodies that contain minimal sequence derived from non-human immunoglobulin (e.g., camelid). For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (sec, e.g., EP 404,097; WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8 (10): 1057-62 (1995); and U.S. Pat. No. 5,641,870).

In some embodiments, the antibodies and CDRs of the present invention can be transferred to another antibody type (e.g., to the heavy chain of antibodies having heavy and light chains) to generate chimeric antibodies. It is intended that the term “antibody type” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2,IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “frame regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, a K_{D} of 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly cross-react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity is measured by incubating a defined amount of the antibody with a defined amount of the antigen under a defined condition and finding the quantity of the antibody bound to the defined amount of antigen. Antibody affinity can be measured by equilibrium dialysis or by the kinetic BIACORE™ method. The dissociation constant, Kd, and the association constant, Ka, are quantitative measures of affinity. The affinity value may be measure in relative ways between two or more antibodies.

In some embodiments, the antibodies described herein or identified according to the methods described herein are blocking antibodies. As used herein, a “blocking” antibody or an antibody “antagonist” is one which inhibits or reduces biological activity of the antigen(s) it binds. In some embodiments, the blocking antibodies or antagonist antibodies or portions completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., U.S. Pat. No. 5,811,097; Deng et al., Blood 92 (6): 1981-1988 (1998); Chen et al., Cancer Res. 58 (16): 3668-3678 (1998); Harrop et al., J. Immunol. 161 (4): 1786-1794 (1998); Zhu et al., Cancer Res. 58 (15): 3209-3214 (1998); Yoon et al., J. Immunol. 160 (7): 3170-3179 (1998); Prat et al., J. Cell. Sci. III (Pt2): 237-247 (1998); Pitard et al., J. Immunol. Methods 205 (2): 177-190 (1997); Liautard et al., Cytokine 9 (4): 233-241 (1997); Carlson et al., J. Biol. Chem. 272 (17): 11295-11301 (1997); Taryman et al., Neuron 14 (4): 755-762 (1995); Muller et al., Structure 6 (9): 1153-1167 (1998); Bartunek et al., Cytokine 8 (1): 14-20 (1996).

In some embodiments, the antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect antibodies that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene or gene signature.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Vector Delivery

The invention also provides a delivery system comprising one or more vectors or one or more polynucleotide molecules, the one or more vectors or polynucleotide molecules comprising one or more polynucleotide molecules encoding an antibody of the present invention.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

### PLATFORMS AND METHODS FOR GENERATING ANTIBODIES

In one aspect, the present invention provides a platform for generating antibodies. The platform is entirely in vitro and allows for efficiently screening and identification of CDRs capable of binding an antigen of interest. The platform provides for libraries of DNA sequences encoding antibodies and methods of generating said libraries. The platform provides for screening the library by ribosome display. The platform provides for identifying families of antibodies capable of binding to an antigen of interest. The platform provides for affinity maturation by mutating selected antibodies. The platform provides for validation of antibodies.

### CeVICA (Cell-Free VHH Identification Using Clustering Analysis)

CeVICA is an integrated platform for in vitro VHH domain antibody engineering distinct that generates CDR-randomized VHH libraries, optimizes ribosome display, can contain a selection cycle with built-in background reduction, and includes a computational approach to perform global binder prediction from post-selection libraries. CeVICA had previously enabled the rapid generation of more than 800 predicted binder families targeting SARS-COV-2 spike receptor binding domain and engineered potent neutralizing antibodies against SARS-COV-2 in response to an ongoing global pandemic caused by the virus (Cohen, 2020; Zhou et al., 2020).

CeVICA leverages the advantages of cell-free display. The process can start with a linear DNA library as input, in which each sequence is unique and encodes for an artificial nanobody with three fully randomized CDRs, and where the 5′ and 3′ ends of the DNA molecules contain elements required for in vitro ribosome display. Next, CeVICA uses ribosome display to link genotype (RNAs transcribed from DNA input library that are stop codon free, and stall ribosome at the end of the transcript) and phenotype (folded nanobody protein tethered to ribosomes due to the lack of stop codon in the RNA). In each selection cycle, the displaying ribosomes bind to an immobilized target, followed by RT-PCR of the RNA attached to the bound ribosomes, which leads to double stranded DNA, which is then in vitro transcribed/translated in a new round of ribosome display. The double stranded DNA in any chosen round is sequenced to obtain full-length nanobody sequences.

CeVICA then groups the sequences into clusters based on similarity of their CDR sequences, such that each cluster represents a unique binding family. One representative sequence from each cluster can be synthesized and characterized for specific downstream applications. The combination of linear DNA libraries, ribosome display, and selection cycles allow display of libraries with much larger diversity (>10¹⁰) than methods depending on cells at similar experimental scale. As selection increases the representation of sequences encoding binders, each binder sequence leads to a cluster of sequences in the output library. Computational clustering following high throughput sequencing identifies them efficiently, promising a more comprehensive view of the landscape of binder potential, as compared to methods that rely on the analysis of individual colonies or sequences (Huo et al., 2020; McMahon et al., 2018).

VHH libraries containing highly random CDRs can be made based on an analysis of natural VHH sequences and using a three-stage PCR and ligation process. First, to guide the VHH library sequence design, the sequence characteristics of many (e.g., around about 300) unique camelid VHHs (representing natural VHHs) from the Protein Data Bank (PDB298) can be analyzed to highlight the three CDR regions, CDR1-3, separated by four regions of low diversity, frame1-4. Analysis of a larger datasets (e.g., containing around about 1,030 sequences from abYsis) show the same sequence features. Consensus sequences can be extracted to design VHH DNA templates encoding the four frames and additional frames can be included to the final mixture of frame templates, based on well-characterized nanobodies. The mixture of VHH frames can serve as templates in PCR reactions, where DNA oligonucleotides with a 5′ NNB sequence can be used to introduce randomization in CDRs, while hairpin DNA oligonucleotides can be used to block ligation of one end of the PCR product. Random amino acids can be introduced for CDR1, for CDR2, and/or for CDR3 to match the most commonly observed CDR lengths in natural VHHs. CDR3s longer than 13 amino acids only account for a minority of natural VHHs (e.g., 36%) and need not be included in the VHH library. CDRs randomized in earlier stages can be subject to duplication in later stages that reduces their diversity. Therefore, CDR2 can be randomized first, followed by CDR1, and then CDR3, which imposes a diversity hierarchy of CDR3>CDR1>CDR2, because this is the overall ranking of diversity that had been observed in CDRs in natural VHHs. The sequence profile of resulting randomized VHH library can meet the design objectives, and largely can mirror the sequence features of natural VHHs. Notably, such library design differs from pervious synthetic nanobody library designs in several keyways: CDR boundaries and length are defined differently (based on the analysis of natural nanobodies. Complete randomization can be performed on all CDR positions with NNB codons (and there is no need to avoid, for example, cysteines in these positions) to maximize amino acid sequence possibilities. Finally, the VHH DNA libraries can contain elements to enable ribosome display, such as an upstream T7 promoter to allow transcription of VHH RNA, a 3xMyc tag, and a spacer downstream of the VHH coding region that stalls peptide release.

The performance of the library in ribosome display can be tested and unproductive sequences can be reduced, such as VHHs that contain frame shifts or carly stops, by ribosome displaying a library only with randomized CDR 1 and CDR2 and performing one round of anti-Myc selection. Functional VHH sequences will express Myc tag at the C-terminal of VHH and are expected to be enriched after anti-Myc selection. A large decrease of unproductive sequences and an increase of full-length VHHs (from 25.3% to 51.9%) can be obtained after anti-Myc enrichment. At the DNA level, there can be an increase of all in-frame CDRI DNA lengths and a decrease of frameshift lengths. The resulting full-length enriched CDR1 and 2 randomized library can be used as PCR template for randomization of CDR3. The final library with all three CDRs randomized (hereafter, “the input library”) can contain a significant proportion of full-length sequences (for example, around about 27.5%), and full-length diversity can be more than 10¹¹ per pg of library DNA.

Binders can be selected by identifing target specific binders by clustering CDR sequences enriched after selection into families, while accounting for sequencing errors. First, the distribution of the sequence match scores can be examined between randomly selected pairs of sequences within a CDR in a library, and these distributions can be compared for each CDR between the input and output libraries. In pre-selection input libraries, the mean match score can be low and the distribution unimodal, as expected given the randomization; whereas after selection, there can be a multi-modal distribution, with one low mode (similar to input) and at least one high mode, which is further distinguished when combining CDR1 and CDR2 match scores. The high mode should reflect binders enriched by the selection rounds. Sequences with a high match score in one CDR can be more likely to have a higher match score in other CDRs. The likely binder sequences can be clustered when exceeding a combined (CDR1+2) match score threshold. This can yield unique clusters for each antigen or binding partner, which some clusters potentially being shared by the two targets. Shared clusters represent background binders and can be excluded from further analysis due to a lack of showing of specific binding to either target. In other words, the shared clusters represent background binders and are excluded from further analysis, because they do not show specific binding to either target.

The input, output and natural CDR sequence distributions can be compared to assess whether starting with a fully random CDR amino acid profile could be generally detrimental to the fitness of binders, and whether selection output mimics a natural amino acid distribution. In natural VHHs, CDR1 and CDR2 can be less diverse than CDR3 with an amino acid profile that favors certain residues, and previous synthetic nanobody library designs sought to recapitulate the CDR1 and CDR2 amino acid preferences of natural nanobodies. However, fully-randomized NNB codons can be used to encode all CDR positions. In principle, such a design might not be ideal if the natural CDR1 and CDR2 amino acid profile is required for functional nanobodies; but it may allow the recovery of possibilities not captured by libraries pre-biased by natural sequence distributions. The output binder CDR profile can be predominantly influenced by the input library rather than by selection towards a natural VHH profile, a natural VHH CDR amino acid profile is not required for VHH binding properties, and a fully random CDR design offers high diversity without a major binding fitness cost (although may have other fitness drawbacks in vivo).

Affinity maturation can effectively improve VHH function. To perform affinity maturation, a critical stage in antibody development in animals, an affinity maturation strategy can be performed based on CeVICA to increase the affinity of target binding VHHs. Error-prone PCR can be used to introduce random mutations across the full-length sequence of various selected VHHs (for example, around 6) and a mutagenized library can be generated. A library size of around about 4.18×10¹⁰ diversity (sufficient to cover the full diversity of VHHs with three mutations per sequence) can be used as input and up to three rounds (or more) of stringent selection can be performed. The libraries can be sequenced pre-and post-affinity maturation, and the mutations in the pre-library and the mutations in the post-library per sequence can be determined. The position-wise amino acid profiles can be calculated and determined for each VHH, and the change in each amino acid proportion at each position can be determined, generating a percent point change table. The putative beneficial mutations can be determined as those with a percent point increase above a set threshold, which can highlight various putative beneficial mutations for each of the selected VHHs. Finally, a list of identified putative beneficial mutations for each VHH can be assembled and incorporated into different combinations of them into each VHH parental sequence to generate multiple mutated variants of each VHH for final assessment.

The potential impact that the VHH sequences may have on immunogenicity in humans can be examined, as a major concern related to the therapeutic use of VHH antibodies is the possibility that, as camelid proteins, they would elicit an immune response. In particular, VHH hallmark residues in frame2 constitute a major difference between camelid VHHs and human VHs. Affinity maturation data can be used to identify potential conversion options for these VHH hallmark residues. It has been found that VHH hallmark residues can be converted to the corresponding human residue as a result of affinity maturation. This implied that at least some of the VHH hallmark residues can be converted to human residues without loss of binding fitness. Such conversions can serve as frame features of VHH library design and improve tolerance of in vitro engineered VHHs by humans. Single domain antibody frames containing all four human hallmark residues have been successfully used for in vitro engineering of single domain antibodies without light chain, demonstrating the feasibility of converting VHH hallmark residues to human residues. Overall, the extension of CeVICA for affinity maturation can offer a strategy for improving antibody function.

True binders and/or neutralizers can also be identified from lower ranked clusters across a full list of clusters. VHHs representing different clusters selected from various locations on the list of clusters ranked by cluster size can be assayed by, for example, ELISA. Positive binders can be identified among many tested VHHs (e.g., around about 78.9% positive rate), further validating the efficacy of the CDR-directed clustering approach for selection of binders.

CeVICA can be used to produce highly potent virus neutralizing agents through iterative optimization.

CeVICA uses NNB codons to randomize CDRs, which may cause over-representation of certain amino acids that could contribute to poor biophysical properties in the output VHHs. The extent of such potential undesired effects can be evaluated by several biophysical assays. Size exclusion chromatography analysis of nanobodies can be performed to show that a majority (e.g., >90%) of the molecules exist as monomers. The impact of cysteines in CDRs on nanobody biophysical properties and function can also be investigated because cysteine can occur at much higher frequencies in the library CDRs than in natural VHH CDRs.

Non-reducing SDS-PAGE gel analysis of VHHs with 0-2 cysteines in their CDRs (using, for example, samples stored at 4° C. for at least 4 weeks) can reveal that VHHs with no CDR cysteine might only have one monomer band, while VHHs with 1 or 2 CDR cysteine might either have a single monomer band or a monomer and a dimer band. Dimers might not be detected in freshly purified samples and might appear overtime at relatively low rate (FIG. 16b). Thus, presence of cysteine in CDRs do not always cause VHH dimers due to disulfide bond formation. The functional consequences of CDR cysteine mediated dimer formation can be evaluated. Older samples, for example around about 7 month-old samples, can show increased signal in ELISA compared to fresh samples and this signal increase can be suppressed by treating with reducing reagent that breaks up disulfide bond. Disulfide bond formation via CDR cysteine does not appear to adversely affect the function of VHHs.

The thermal stability of VHHs produced by CeVICA can also be assessed. VHHs can show good resistance to thermal denaturation and have a melting temperature of, for example, around about 72° C., which is comparable to VHHs generated by other methods. The ability to refold after complete thermal denaturation can be compared by ELISA readings of VHH samples before and after heating at 98° C. for 10 minutes. A heated/non heated ratio greater than 1, indicating increased binding affinity after complete thermal denaturation and refolding, which could result from expedited disulfide bond formation that may increase the percentage of dimers in the samples subjected to heating. This hypothesis is supported by the observation that samples heated and refolded in the presence of reducing reagent can have a heated/non heated ratio of 1. Thus, VHHs produced by CeVICA can have good thermal stability and can efficiently refold after complete thermal denaturation.

The CeVICA platform offers a generalizable solution for in vitro VHH antibody engineering that integrates all the components necessary to generate VHH binder sequences in a cell-free process. CeVICA VHH libraries are designed to contain only the essential features for robust VHH structure, revealed by the diversity profile across the length of natural VHHs. Fully random NNB encoded codons in all CDR positions have been validated to not adversely affect binder selection or impact biophysical stability of individual VHHs produced by the platform. A linear DNA library of such design can be efficiently produced by a method of successive PCR and ligation, yielding large libraries with their library size directly quantifiable. This library generation method is highly adaptable, for example, oligos containing alternative base mix ratios can be used to achieve different amino acid profiles for specific CDR positions, alternative frame template sequences can be used to enrich for unique biophysical properties encoded in the frame regions of VHHs. Lastly, these linear libraries perform well when used as input to an optimized ribosome display based selection protocol, which suppresses sequence segment shuffling that could break up CDR pairing, a challenging problem often associated with cell free systems.

A key feature of CeVICA is binder sequence recover using CDR-directed clustering. This approach fully utilizes all sequences in the output library to provide a comprehensive view of all binders contained in the output and, in effect, reduces the VHH characterization screen space. This feature makes CeVICA particularly suited for applications where large numbers of antibodies need to be screened to isolate ones with unique traits (e.g., virus neutralization, receptor activation, targeting hard-to-target epitopes) in addition to binding the target. Without computational clustering, it can be difficult to recover VHHs with specific traits by random sampling.

Previous synthetic nanobody library designs sought to randomize CDR positions using an amino acid profile that recapitulates the profile observed in the corresponding positions in natural nanobodies; however, whether the natural profile represents an ideal profile for the purpose of in vitro antibody engineering is not certain. The large number of nanobody clusters that can be generated using CDR-directed clustering offers the opportunity to test the fitness of randomized amino acid profile in binder selection. In many positions, the output binder profile can highly resemble the input library profile, while the similarity between the output profile and the natural profile can be lower. For positions where the output profile moves significantly away from the input profile, the distance between output and natural profiles can be greater than that between output and input, and also greater than the distance between input and natural, which can indicate that the output profile is not moving closer to a natural profile in these positions. The amino acid profiles observed in natural nanobodies are not necessarily more fit than an NNB profile for binder selection (although they may be more fit for other features). A strategy to improve the fitness of an input library can be to incorporate amino acid profiles that match the output profile, which can be achieved by using specifically defined (non-equal) base mix ratios for the three base positions of a randomizing codon. Such a strategy could provide improvements on synthetic nanobody library design.

VHHs produced by CeVICA can show good biophysical properties that are comparable to VHHs of animal origin. For example, robust refolding after complete thermal denaturation up to 100% can be seen. Such high refolding capability may be partly explained by the use of ribosome display for the selection of VHHs, during which VHHs need to fold into their functional confirmation while tethered to ribosomes in a minimally reconstituted protein synthesis environment that lacks factors normally found inside cells to aid protein folding such as chaperons, thus enriching for VHHs with strong inherent folding stability. This hypothesis could be tested further as CeVICA gets applied to more cases. CeVICA's suitability for engineering high affinity VHH antibodies with comparable biophysical properties as VHHs produced by animals has been demonstrated, making it a valuable addition to in vitro antibody engineering technologies.

In conclusion, CeVICA is a system for synthetic VHH based antibody library design, in vitro selection optimization, post-selection screening, and affinity maturation. Using CeVICA, large collections of antibodies that can bind the antigens can provide an important resource. Given its seamlessly integrated procedure, CeVICA is amenable to automation and provides an important tool for antibody generation in a rapid, reliable and scalable manner. CeVICA further provides a technology framework for incorporation of refinements that could overcome limitations of in vivo fitness of in vitro generated antibodies and the overall efficiency of cell-free antibody engineering.

For example, but not meant to be limiting, nanobodies (VHHs) can be separated into CDRs and frames (segments) by finding regions of continuous sequence in each VHH that best matched to the following standard frame sequences: frame1 standard:

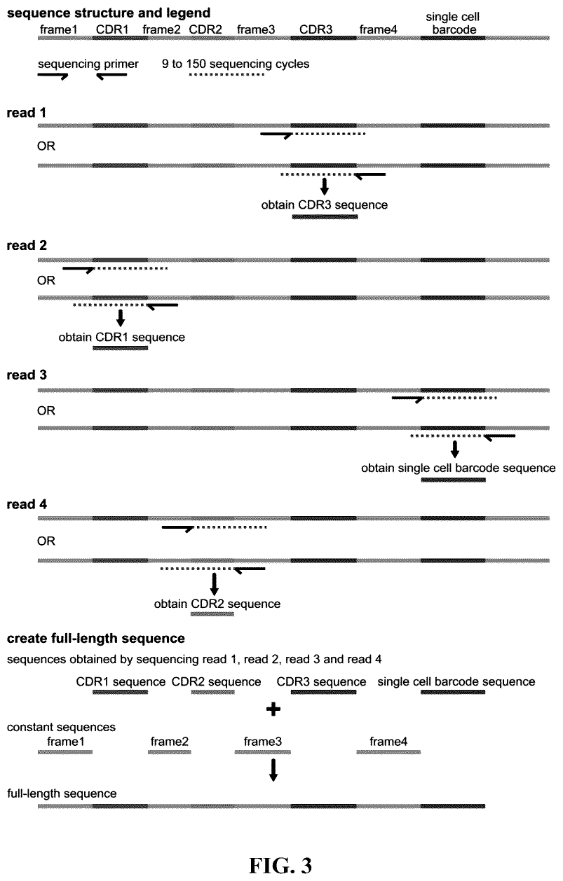

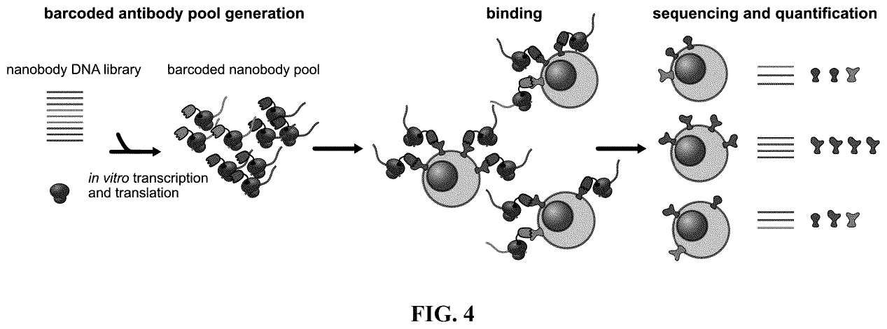

EVOLVESGGGLVQAGDSLRLSCTASG (SEQ ID NO: 13102), frame2 standard: MGWFRQAPGKEREFVAAIS (SEQ ID NO: 13103), frame3 standard: AFYADSVRGRFSISADSAKNTVYLQMNSLKPEDTAVYYCAA (SEQ ID NO: 13104), frame4 standard: DYWGQGTQVTVSS (SEQ ID NO: 13105). Each matched region is the corresponding frame of the VHH, the region between frame1 and frame2 is CDR1, the region between frame2 and frame is CDR2, the region between frame3 and frame4 is CDR3 (see FIG. 3). Only nanobody sequences with at least one unique CDR can be selected to represent natural nanobodies and used for constructing amino acid profile (a.a. profile). 298 sequences from Protein Data Bank (PDB298) and 1,030 sequences from abYsis (abYsis1030) can fit this selection criteria. The amino acid (a.a.) profile at each position within each segment can be calculated by finding the percentage of each of the 20 universal proteinogenic amino acid at that position among all selected VHHs, all frame lengths can be set to the same length as frame standards. CDR lengths can be manually set to accommodate different CDR lengths, CDR1 and CDR2 lengths can be set to 10, CDR3 length can be set to 30. VHHs with CDR lengths shorter than the corresponding set length can have their CDR filled from the C-terminal end with empty position holders up to the set length. CDR boundaries can be defined by the position where the combined frequency of the top two most abundant amino acids dropped sharply.

The above referenced annotation method, and the methods according to Kabat and Chothia (www.abysis.org/abysis/sequence_input/key_annotation/key_annotation.cgi) have been compared and all three methods (Kabat, Chothia and the above) showed frame regions with the same core sequence, and with 1-2 amino acid differences in the exact CDR boundaries between the three methods. The performance of the above-referenced library suggests their annotation faithfully captured the domain structure of nanobodies.

VHH library construction. Nanobody library sequence can be designed to recapitulate the sequence structure of frames and CDRs observed from analyzing natural nanobodies (see PDB298, abYsis1030). The design differs from prior designs in both the length of CDRs and the positions selected for randomization and randomization strategy. Such differences likely arise from differences in the size of natural nanobody collection retrieved from databases and/or in how the nanobodies are annotated and analyzed (Amino acid profile construction and analysis of natural nanobodies). For example, the analysis showed the percentage of nanobodies containing CDR2 with lengths 4, 5, or 6 amino acids (a.a). are 32%, 61%, and 1.7% respectively, so CDR2 with a length of 5 a.a. can be chosen to recapitulate the most prevalent CDR2 length.

VHH libraries can be constructed by ligation of PCR products in three stages, with each stage randomizing one of the three CDRs. Primers can be synthesized using standard DNA oligo synthesis and purified by desalting without PAGE purification, The level of synthesis errors with standard oligo synthesis and desalting purification may not have significant impact on the functionality of the nanobody library. At each stage, PCR can be performed using a high-fidelity DNA polymerase without strand displacement activity, using Phusion DNA polymerase (New England Biolabs, M0530L). 65° C. can be used as the elongation temperature to avoid hairpin opening during DNA elongation. PCR products with correct size can be purified by DNA agarose gel extraction. Ligation and phosphorylation of PCR products can be performed simultaneously using T4 DNA ligase (New England Biolabs, M0202L) and T4 Polynucleotide Kinase (New England Biolabs, M0201L). Ligation products with the correct size can be purified by DNA agarose gel extraction using NucleoSpin Gel and PCR Clean-Lip Kit (Takara, 740609.250). Purified ligation products can be quantified with Qubit IX dsDNA HS Assay Kit (ThermoFisher Scientific, Q33230 using Qubit 3 Fluorometer.

CDR2 can be randomized in stage one, PCR templates with equal molar mixtures of plasmids carrying DNA encoding frames, including three frame 1 versions, one frame2, three frame3 versions and one frame4. The three versions of framel and frame3 can be derived from consensus sequence extracted from natural VHH a.a. profiles. CDRI can be randomized in stage two: 200 ng of ligation product from the first stage can be digested by Notl-HF (New England Biolabs, R3189S) and heat denatured. The entire digestion product can be used as template for PCR in stage two. Ligation product of stage two can be subject to one round of ribosome display and anti-Myc selection (below), the entire recovered RNA can be reverse transcribed and PCR amplified and purified.

270 ng of this RT-PCR product can be used as template for PCR in stage three to randomize CDR3. Ligation product of stage three can be purified by DNA agarose gel extraction. The purified ligation product can then be digested by Dral (New England Biolabs, R0129S) and a fragment of −680 bp in size can be purified by DNA agarose gel extraction to get the final VHH library, referred to as the input library.

High throughput full-length sequencing of VHH library. Sequencing libraries from VHH DNA libraries can be prepared by two PCR steps using primers and PCR cycling conditions. Equal mixtures of Phusion DNA polymerase (New England Biolabs, M0530L) and Deep Vent DNA polymerase (New England Biolabs, M0258L) can be used for both PCRs to ensure efficient amplification. PCR cycle number can be chosen to avoid over-amplification and typically falls between 5 to 15.

In the first PCR, Illumina universal library amplification primer binding sequence and a stretch of variable lengths of random nucleotides can be introduced to the 5′ end of library

DNA. Similarly, Illumina universal library amplification primer binding sequence and a stretch of variable lengths of index sequence can be introduced to the 3′ end of library DNA. Eight different lengths can be used for both random nucleotides and index to create staggered VHH sequences in the sequencing library, this arrangement can be required for high quality sequencing of single amplicon libraries on an Illumina Miseq instrument. The product of the first PCR can be purified by column clean-up using NucleoSpin Gel and PCR Clean-Up Kit and the entire sample can be used as template for the second PCR.

In the second PCR, Illumina universal library amplification primers can be used to generate sequencing library. Sequencing libraries can be purified by DNA agarose gel extraction, quantified using Qubit 3 Fluorometer, and sequenced on an Illumina Miseq instrument using MiSeq Reagent Nano Kit v2 (500-cycles) (Illumina, MS-103-1003). Sequencing run setup can be: paired end 2×258 with no index read. Index in the library can be designed as inline index, so a separate index read might not be required. Raw reads can be separated by index, trimmed to remove N bases and bases with a quality score of less than 10 prior to downstream analysis.

Ribosome display. VHH DNA library containing a specified amount of diversity can be first amplified using a DNA recovery primer pair. Equal mixtures of Phusion DNA polymerase (New England Biolabs, M0530L) and Deep Vent DNA polymerase (New England Biolabs, M0258L) can be used for the PCR. PCR cycle number can be chosen to avoid overamplification and typically falls between 5 and 15. In a standard preparation, 200-500 ng of the purified PCR product can be used as DNA template in 25 pl of coupled in vitro transcription and translation reaction using PURExpress In Vitro Protein Synthesis Kit (New England Biolabs, E6800L). The reaction can be incubated at 37° C. for 30 minutes, then placed on ice, and 200 pl ice cold stop buffer (10 mM HEPES pH 7.4, 150 mM KCl, 2.5 mM MgCh, 0.4 pg/pl BSA (New England Biolabs, B9000S), 0.4 U/pl SUPERascHn (ThermoFisher Scientific, AM2696), 0.05% TritonX-100) can be then added to stop the reaction. This stopped ribosome display solution can be used for binding to immobilized protein targets during in vitro selection. The amount of DNA template, volume of coupled in vitro transcription and translation reaction, and volume of stop buffer were scaled proportionally when different volumes of stopped ribosome display solution can be needed. 1 to 8× standard preparations can be used for each selection round with the first round using 8× standard preparations, second round using 2× standard preparations and third round using IX standard preparation.

In vitro selection. Target proteins can be immobilized to magnetic beads by first coating protein G magnetic beads (ThermoFisher Scientific, 10004D) with anti-Flag antibody (Sigma-Aldrich, Fl 804), then incubating antibody-coated beads with cell lysate or cell media containing 3xFlag tagged target proteins at 4° C. for 2 hours. For anti-Myc selection, magnetic beads can be coated by anti-Myc antibody (ThermoFisher Scientific, 13-2500) only. 100 pl of beads can be used for the first round of selection, and 50 pl of beads can be used for subsequent rounds. The beads can be washed three times with PBST (PBS, ThermoFisher Scientific, with 0.02% TritonX-100). Stopped ribosome display solutions can be first incubated with antibody-coated beads (without targets) at 4° C. for 30 minutes for pre-clearing of non-specific and off-target binders, the solution can then be transferred to target immobilized beads and incubated at 4° C. for 1 hour, the target immobilized beads can then be washed 4 times with wash buffer (10mM HEPES pH 7.4, 150 mM KCl, 5 mM MgCh, 0.4 pg/pl BSA (New England Biolabs), 0.1U/pl SUPERascHn (ThermoFisher Scientific), 0.05% TritonX-100). After washing, beads can be resuspended in TRIzol Reagent (ThermoFisher Scientific, 15596026), and RNA can be extracted from the beads, 25 pg of linear acrylamide (ThermoFisher Scientific, AM9520) can be used as co-precipitant during RNA extraction. Reverse transcription of extracted RNA can be performed using Maxima H Minus Reverse Transcriptase (ThermoFisher Scientific) and primers. The reverse transcription reaction can be purified using SPRIselect Reagent (Beckman Coulter) to obtain purified cDNA. Purified cDNA can be amplified by PCR using equal mixtures of Phusion High-Fidelity DNA polymerase and Deep Vent DNA polymerase. PCR cycle number (Table 12) can be chosen to avoid over-amplification and typically falls between 10 to 25. This PCR condition ensures efficient full-length product synthesis at each cycle and is required to faithfully amplify nanobody genes without CDR shuffling, a phenomenon that could otherwise cause selection failure. The PCR product can be purified by DNA agarose gel extraction. The purified PCR product can be used for library generation for high throughput full-length sequencing or as DNA input for ribosome display reaction (coupled in vitro transcription and translation) to perform additional rounds of in vitro selection.

One round of anti-Myc selection can be performed on the nanobody library with CDR1 and 2 randomized to enrich for correct-frame sequences. Several factors can in principle contribute to the presence of out-of-frame sequences after anti-Myc selection: (1) Non-specific binding of RNA or protein to magnetic beads; (2) translation through alternative start codons downstream of areas containing out-of-frame errors; and/or (3) inefficient binding of anti-Myc antibody to the expressed Myc peptide that is located between the VHH protein and ribosome. Option 1 is disfavored, because although their input library contained 27.5% full-length sequences, the remaining sequences that contained errors do not interfere with full-length sequences and are reduced to <10% after three rounds of RBD selection, suggesting that erroneous sequences or their encoded peptides do not non-specifically stick to beads at significant levels to impact binder selection.

CDR-directed clustering analysis. Computational analysis for CDR-directed clustering can be performed using custom python scripts. Paired end sequences can be merged to form full-length VHH sequences. Merged VHH sequences can be quality trimmed and translated into VHH protein sequence, which can be separated into CDRs and frames (segments) as described above. Two VHHs can be determined to have similar CDRs via the following steps. First, the ungapped sequence alignment score (match score) can be calculated for each CDR of the two VHHs as the sum of BLOSUM6230 amino acid pair scores at each aligned position. (If two CDRs have different lengths, their sequence alignment score can be set to −5 by default.) The alignment scores of any two pairs of CDRs can be summed to yield three scores, and if at least one of the three is larger than 35, the two VHHs can be defined as having similar CDRs. Next, VHHs with similar CDRs can be grouped by a two-step process. In the first step, VHH cluster-forming “seeds” can be chosen whose VHHs that were called as similar to at least 5 other VHHs (all remaining VHHs were not considered for clustering). In the second step, a seed VHH was iteratively selected with at least 5 other similar (>35 match score) seed VHHs, and grouped all of them into one cluster, removing them from the seed VHH pool, and iterated this procedure until no seed VHHs remained. For example, for one antigen, there can be 83,433 seeds in the first step, and 83,392 can be grouped in clusters in the second step. For a different antigen, 71,210 of 71,220 seeds can be grouped in clusters. This heuristic was fast in a standard computing environment with multiprocessing capabilities.

A representative sequence to illustrate each CDR in each cluster can be chosen as the most frequent CDR sequence in the cluster (the chosen representatives for CDR1,2, and 3 may not necessarily be from the same sequence, and were used only for illustrative purposes for each cluster; whole VHH sequences can be used for gene synthesis and all downstream experiments). A consensus sequence can be generated for each CDR, where each position in the CDR can be represented by a 6 character string, such that the first and fourth character can be the single letter code for the top and the second most abundant amino acid at the position, respectively, and the following two characters (second and third for the most abundant; fifth and sixth for the second most abundant), can be their frequency, respectively (ranging from 00 for <34% to 99 for 100%). The consensus sequence for a CDR can be recorded as a single “BOO” when the standard deviation of the lengths of all CDRs was greater than 1. CDR scores can be calculated by summing a score for each position in the CDR consensus sequence, with scores of 3, 2, 1 for positions where the most abundant amino acid had frequencies greater than 80%, 50%, or less, respectively, and a score of 0 for CDRs with a consensus sequence of a single “BOO”. Representative whole nanobody sequence for each cluster can be selected as the one with the maximal sum (max−sum) of all CDR similarity score between the nanobody and all other nanobodies in the cluster. This max-sum representative nanobody sequence selection process minimizes the impact of random errors introduced during NGS library preparation and sequencing by imposing a scoring penalty on sequences containing random errors.

Mean distance to diagonal can be calculated based on squares of residuals, where the residual=y−x, the distance to diagonal (the half diagonal length of the square of residual)=\hat{ }square of residual/2. The mean distance to diagonal represents the average distance of data points to the diagonal line in a scatter plots of two amino acid profiles and is a measure of the difference between the two amino acid profiles.

Affinity maturation. Error-prone PCR can be used to introduce random mutations across the full length of selected VHH DNA sequences. 0.1 ng of plasmid carrying DNA sequence encoding each selected VHH can be used as template in PCR reactions using Taq DNA polymerase with reaction buffer (10 mM Tris-HCl pH 8.3, 50 mM KC1, 7 mM MgCh, 0.5 mM MnCh, 1 mM dCTP, 1 mM dTTP, 0.2 mM dATP, 0.2 mM dGTP) suitable for causing mutations in PCR products. Mutagenized library for input to CeVICA can be made by ligating PCR products of error-prone PCR that carries VHH to DNA fragment containing the remaining elements required for ribosome display. Three rounds of ribosome display and in vitro selection can be performed on the mutagenized library (pre-affinity maturation, after error-prone PCR) as described in the In vitro selection section, during which the incubation time of the binding step can be kept between 5 seconds to 1 minute to impose a stringent selection condition, additional error-prone PCR can be not performed during the selection cycles. The output library (post-affinity maturation) can be sequenced along with the pre-affinity maturation library as described in the High throughput full-length sequencing of VHH library section.

In some embodiments, the platform utilizes a VHH library obtained by methods described further herein. In some embodiments, the platform utilizes VHH frameworks randomized at all CDRs in the framework (e.g., CDR1, CDR2, and CDR3). Libraries can include a varying number of members, such as up to about 100 members, such as up to about 1,000 members, such as up to about 5,000 members, such as up to about 10,000 members, such as up to about 100,000 members, such as up to about 500,000 members, or even more than 500,000 members. In one example, the methods can involve providing a VHH library containing a large number of potential antibodies. Such libraries cen then screened by the methods disclosed herein to identify those library members that display a desired characteristic activity (e.g., binding).

Identification and ranking of beneficial mutations. To identify potential beneficial mutations for each selected VHH an amino acid profile (a.a. profile) table can be built for each VHH family in the pre-and post-affinity maturation library, and identified amino acids with increased frequency in the post-affinity maturation population compared to their pre-maturation frequency. For each VHH parental sequence, an a.a. profile can be built of the percent of each a.a. across all VHH sequences originated from one parental VHH in the pre-affinity maturation library (“pre-a.a. profile”) and in the post-affinity maturation library (“post-a.a. profile”). A percent point change table can be generated by subtracting the pre-a.a. profile from the post-a.a. profile, describing the change of frequency of each observed amino acid at each position of the VHH protein following affinity maturation.

A putative beneficial mutation can be defined as either (1) the non-parental amino acid with the biggest increase in frequency if its increase is at least 0.5 percentage points; the score is the difference from the parental amino acid frequency; or (2) the non-parental amino acid with the biggest increase after the parental amino acid if the increase is at least 1.5 percentage points; the score is the percent point change of the beneficial mutation. To avoid too many proximal putative beneficial mutations (which may cause structural incompatibility), a putative beneficial mutation can be discarded if it (1) is outside the CDRs; (2) is less than 3 positions away from another beneficial mutation (“nearby mutation) and has a smaller beneficial mutation score than the nearby mutation; and (3) co-occurs less than twice with the nearby mutation. From this final list of putative beneficial mutations, different combinations can be picked and incorporated into each VHH parental sequence that include one combination of all beneficial mutations in CDRs, one combination of the top-3 ranked (by beneficial mutation score) mutations in frames, and at least one combination of both CDR mutations and frame mutations.

In some embodiments, the library is generated by analyzing naturally occurring antibody frameworks (e.g., camelid heavy chain antibodies). Templates are then generated with selected frameworks. In some embodiments, frameworks are chosen having the most variation between the frameworks. Sets of primer pairs are generated to randomly mutate each CDR sequence in each framework. Each CDR is randomized with a set of two pairs of primers corresponding to the entire framework sequence. For example, the first pair of primers amplifies a first half of the framework and the second pair of primers amplifies the second half of the framework directly adjacent to the start of the first amplicon. The set of primers for the first CDR can include primers hybridizing to each end of the framework (i.e., the first pair and second pair of primers each includes a primer specific to one end). The primers specific to the ends of the framework are preferably blocked from being ligated. In some embodiments, the primers are blocked by including a hairpin sequence. The primers for randomizing in each primer pair (i.e., not the primers hybridizing to the ends) hybridize to a region that is not mutagenized and include a randomized sequence. The region for hybridization is selected such that the primer hybridizes under the PCR annealing conditions (e.g., 50-70° C.). The primers also include a randomized sequence corresponding to the number of amino acids in the region of the CDR to be mutagenized. Randomization schemes may include NNN, which uses all 64 codons; NNB, which uses 48 codons; NNK, which uses 32 codons; and MAX, which assigns equal probabilities to each of the 20 amino acids (where N=A/C/G/T, B=C/G/T, S=C/G, and K=G/T) (see, e.g., Nov, Y., Appl Environ Microbiol. 2012 January; 78 (1): 258-262). In some embodiments, randomization schemes are used that avoid stop codons.

In some embodiments, the library is generated by using PCR and ligation for each CDR in the framework. In some embodiments, the PCR primer pairs in each set generate two amplicons capable of being ligated in only one orientation due to the ends of the amplicon being blocked. The ligated PCR product from the previous step is used as the template for the successive CDR randomization steps. The randomized sequence may be present in one or both of the primer pairs for a CDR sequence (e.g., CDR3). In some embodiments, the PCR for each randomization step is performed using a DNA polymerase without or having extremely weak strand displacement activity (e.g., Phusion® High-Fidelity DNA Polymerase, New England Biolabs). The term strand displacement describes the ability to displace downstream DNA encountered during synthesis, such as a downstream double stranded region (e.g., hairpin). The terms “weak” and “strong” relate to the strength of displacement as compared to an average activity. In some embodiments, weak refers to an activity that is only slightly greater than no activity. In some embodiments, weak displacement activity refers to less than 95% or 99% of an encountered DNA strand is displaced and strong displacement activity refers to greater than 95% or 99% of encountered DNA is displaced under normal reaction conditions. In some embodiments, the CDR with the shortest sequence is randomized in the first or second cycle and the CDR with the longest sequence is randomized in the last step (e.g., CDR3).

In some embodiments, the framework sequence includes a promoter sequence. The promoter sequence is preferably compatible with in vitro transcription/translation systems (e.g., T7 promoter). In some embodiments, the promoter is a constitutive promoter, a native promoter, an inducible-promoter, or tissue-specific promoter. In some embodiments, the framework sequence includes an enhancer. Thus, in some embodiments, the library is transcribed into mRNAs encoding for each antibody framework. The mRNA can then be translated to generate the antibody polypeptide. In other embodiments, the library comprising mRNAs encoding each antibody framework are not transcribed in vitro before in vitro translation and are instead harvested from host cells or chemically synthesized by methods known in the art. In some embodiments, library members are cloned into vectors including the promoter sequence and/or enhancer sequence. In some embodiments, an individual vector may comprise one or more library members. In some embodiments, an individual vector map comprise two, three, four, five, six, seven, eight, or more library members In some embodiments, a vector comprising multiple library members may comprise one promoter and/or enhancer sequence that regulates the expression of the library members encoded therein. In some embodiments, a vector comprising multiple library members may comprise multiple promoter and/or enhancer sequences that regulates the expression of the library members encoded therein. In some embodiments, nucleic acids encoding library members may comprise one or more chemically modified moieties.

In some embodiments, the framework sequence does not include a stop codon, whereby the mRNA and translated protein is not released by ribosomes. In some embodiments, the platform includes ribosome display (see, e.g., Zahnd, et al., Ribosome display: selecting and evolving proteins in vitro that specifically bind to a target. Nat Methods 4, 269-279 (2007)). As used herein ribosome display refers to an in vitro selection and evolution technology for proteins and peptides from large libraries. In some embodiments, the antigens of interest (e.g., cell surface proteins) are provided on one or more surfaces which bind the one or more of said antigens of interest and display them on the surface. In some embodiments, the antigens of interest (e.g., cell surface proteins) are provided by one or more target cells which express one or more of said antigens of interest and display them on the cell surface. In some embodiments, the antigens of interest (e.g., cell surface proteins) are provided as individual protein molecules which freely circulate in solution and not bound or physically associated with anything until they bind to library members. In some embodiments, antigens of interest (e.g., cell surface proteins) which are provided in a solution not bound or physically associated with anything are captured after binding to library members by inducing attachment of the antigen of interest to a solid surface (e.g., a bead). In some embodiments, the antigen of interest (e.g., cell surface proteins) is immobilized to a solid surface (i.e., surface-immobilized target), such as magnetic particles, latex beads, nanoparticles, macro-beads, membranes, microplates, array surfaces, dipsticks, hydrogels, and a host of other devices that facilitate the capture of specific biomolecules. The surfaces are then used to select for translated antibody frameworks capable of binding the antigen of interest. The surfaces can be washed and mRNA can be isolated. The mRNA can be converted to cDNA by reverse transcription PCR (RT-PCR). In preferred embodiments, the PCR reaction in the RT-PCR is performed using a mixture of two DNA polymerases, wherein one type is a DNA polymerase without or having extremely weak strand displacement activity (e.g., Phusion® High-Fidelity DNA Polymerase, New England Biolabs) and the other type is a DNA polymerase with strong strand displacement activity (e.g., Deep Vent® DNA Polymerase, New England Biolabs). The cDNA can then be used as input for successive rounds of ribosome display. In preferred embodiments, 3 rounds are performed, however 1, 2, 3, 4, 5, 6, 7, 8, 9 or 10 or more rounds can be performed. In some embodiments, the number of rounds is saturated when the same library members are isolated at each round.

In some embodiments, the stringency of the washing and binding steps is adjusted to increase binding stringency. In some embodiments, stringency is increased by increasing the ionic strength of the buffers. In some embodiments, stringency is increased by adding or increasing the concentration of detergent in the buffers. The binding and washing is preferably performed at about 4° C., however stringency can be changed by increasing the temperature. In some embodiments, binding time is adjusted. In some embodiments, binding time in initial cycles of ribosome display may be longer and in successive cycles decreased to increase stringency. For example, antibodies that bind overnight can be identified in an early round. In some embodiments, binding is performed overnight (about 12 hours), 4 hours, 3, hours, 2 hours, 1 hour, or less than 1 minute. In some embodiments, binding is performed in buffers containing Mg²⁺ions at concentrations of 5 mM or less.

In some embodiments, library members are cloned into vectors including an epitope tag sequence. In some embodiments, the framework includes a sequence encoding for an epitope tag in frame with the antibody sequence and at the C-terminus of the antibody. In some embodiments, the framework includes a sequence encoding for an epitope tag in frame with the antibody sequence and at the N-terminus of the antibody. In some embodiments, the N-terminal or C-terminal tag is hemagglutinin (HA). In some embodiments, the N-terminal or C-terminal tag is FLAG. In some embodiments, the N-terminal or C-terminal tag is one, two, three, four, five, or six instances of HA (referred to as, in the example of 6 instances of HA, either 6X-HA, 6HA, or 6-HA). In some embodiments, the N-terminal or C-terminal tag is one, two, or three instances of FLAG (referred to as, in the example of 3 instances of FLAG, 3X-FLAG, 3FLAG, or 3-FLAG). In some embodiments, the N-terminal or C-terminal tag is GST, Myc, polyHis, TAP, V5, biotin, MBP, or SpyTag. In some embodiments, the N-terminal or C-terminal tag is one, two, three, four, five, six, seven, eight, or nine instances of Myc. In some embodiments, the N-terminal or C-terminal tag is one, two, three, four, five, six, seven, eight, nine, ten, eleven, or twelve instances of His. In some embodiments, the N-terminal or C-terminal tag is one, two, or three instances of V5. In some embodiments, the epitope tag can be used to enrich for full length mRNA sequences. For example, the entire antibody with the epitope tag is encoded by only a full-length mRNA. Thus, enriching for ribosomes expressing an antibody framework fused to an epitope tag will enrich for full-length mRNAs. In some embodiments, enrichment is performed for one or more rounds. In some embodiments, full-length mRNA are enriched during the step of generating the library. In some embodiments, the first or first and second CDRs are randomized. The randomized frameworks are then used in ribosome display followed by enrichment using a solid surface specific for binding to the epitope tag. The enriched mRNA are then converted to cDNA and used as input for randomizing the last CDR sequence.

Methods of enriching for antibody frameworks comprising a tag in complex with ribosomes bound to mRNAs corresponding to said antibody framework will be well available to one of ordinary skill in the art. For example, in some embodiments, epitope tagged-antibody frameworks in complex with ribosomes bound to mRNAs corresponding to the frameworks may be enriched by incubating said antibody frameworks with antibodies against the respective epitope tag. Antibodies against tags including HA, FLAG, Myc, V5, GST, TAP, MBP, and SpyTag are commercially available and may be conjugated to solid support such as beads and other affinity column materials. Alternatively, polyHis-tagged proteins can be isolated using Ni-NTA agarose beads and affinity columns comprising said beads. In further alternative approaches, biotin-tagged proteins may be isolated by incubation with solid supports (e.g., beads) bound to streptavidin. Similarly, various methods for validating enrichment of antibody frameworks and complexes thereof include western blot, flow cytometry, mass spectrometry, and enzyme-linked immunosorbent assay (ELISA).

The platform also includes computational steps capable of clustering antibodies having similar CDRs. In some embodiments, clustering allows for families of related antibodies to be identified, such that a few antibodies from each family can be further assayed or validated to determine binding or neutralizing activity of different families. In some embodiments, every family identified is further validated. In some embodiments, 1, 2, 3, 4, 5, 6, 7, 8, 9, 10 or 20 or more antibodies clustered in a family are validated.

In some embodiments, antibody frameworks identified using a randomized library are further mutated by one or more rounds of affinity maturation. Affinity maturation refers to the introduction of random mutations across the full length of selected VHH DNA sequences (i.e., also including frame regions) and performing ribosome display with the antigen of interest. In some embodiments, binding during ribosome display is performed for 1 minute or less.

In some embodiments, the platform includes assays capable of validating antibody binding and neutralization activity. In some embodiments, the validation assay is an immunoassay. Immunoassay methods are based on the reaction of an antibody to its corresponding target or analyte and can detect the analyte in a sample depending on the specific assay format. Immunoassays have been designed for use with a wide range of biological sample matrices. Immunoassay formats have been designed to provide qualitative, semi-quantitative, and quantitative results. Quantitative results may be generated by determining the concentration of analyte detected by an antibody.

Numerous immunoassay formats have been designed. Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like. ELISA, or enzyme immunoassay (EIA) can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Viral neutralizing assays can be used to validate identified antibody frameworks. In some embodiments, the assay uses live virus. In some embodiments, the assay uses pseudotyped virus particles (see, e.g., Gentili, 2015). In some embodiments, the neutralizing assay is performed without live virus (sec, e.g., Tan, C. W., Chia, W. N., Qin, X. et al. A SARS-COV-2surrogate virus neutralization test based on antibody-mediated blockage of ACE2—pike protein—protein interaction. Nat Biotechnol 38, 1073-1078 (2020).

Various embodiments are described herein. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Furthermore, the particular features, structures or characteristics may be combined in any suitable manner, as would be apparent to a person of ordinary skill in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

Further embodiments are illustrated in the following Examples which are given for illustrative purposes only and are not intended to limit the scope of the invention.

### Compositions and Methods

In some aspects, the present disclosure provides a variety of compositions comprising antibodies or antigen-binding fragments thereof derived from the approaches described herein. In some aspects, the present disclosure also provides a variety of methods (e.g., methods of treating a subject) involving the use of antibodies or antigen-binding fragments thereof derived from the approaches described herein.

Compositions comprising an antibody or antigen-binding fragment thereof described herein may further optionally comprise a liposome, a lipid, a lipid complex, a lipid nanoparticle, a microsphere, a microparticle, a nanosphere, or a nanoparticle, or may be otherwise formulated for administration to the cells, biological samples, tissues, organs, or body of a subject in need thereof.

Compositions comprising an antibody or antigen-binding fragment thereof described herein may further comprise a pharmaceutical excipient. Pharmaceutically acceptable excipients (excipients) are substances other than the therapeutic agent that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, or delivery of the therapeutic agent during storage or use. A pharmaceutically acceptable excipient may or may not be an inert substance. Excipients include, but are not limited to, absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions comprising an antibody or antigen-binding fragment described herein can contain other additional components commonly found in pharmaceutical compositions. Such additional components can include, but are not limited to: anti-pruritics, astringents, local anesthetics, or anti-inflammatory agents (e.g., antihistamine, diphenhydramine). In fact, there is virtually no limit to the components of a composition comprising an antibody or antigen-binding fragment described herein may also include any of those that are Generally Recognized as Safe (GRAS) by the United States Food and Drug Administration.

Compositions comprising an antibody or antigen-binding fragment described herein may be suitable for treatment regimens and thereby administered to a subject via a variety of methods described herein. Such compositions may be formulated for use in a variety of therapies, such as, for example, in the amelioration, prevention, and/or treatment of conditions. Accordingly, the compositions comprising an antibody or antigen-binding fragment thereof described herein may be administered to a subject such as human or non-human subjects, a host cell in situ in a subject, a host cell ex vivo, a host cell derived from a subject, or a biological sample (e.g., one derived from a subject).

In some embodiments, administration of an antibody or antigen-binding fragment thereof described herein achieves one, two, three, four, or more of the following effects, including, for example: (i) reduction or amelioration the severity of disease, disorder, or condition or symptom associated therewith; (ii) reduction in the duration of a symptom associated with a disease, disorder, or condition; (iii) protection against the progression of a disease or disorder or symptom associated therewith; (iv) regression of a disease, disorder, or condition or symptom associated therewith; (v) protection against the development or onset of a symptom associated with a disease, disorder, or condition; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease, disorder, or condition; (xi) an enhancement, improvement, supplementation, complementation, or augmentation of the prophylactic or therapeutic effect(s) of another therapy.

Sterile injectable solutions comprising an antibody or antigen-binding fragment thereof described herein are prepared by solvation in the required amount of appropriate solvent in addition to any of the other ingredients enumerated above, as required, followed by filtered sterilization. Generally, dispersions are prepared by incorporating the various sterilized ingredients into a sterile vehicle that contains the basic dispersion medium and the other ingredients from those enumerated above.

For administration of an injectable aqueous solution, the composition comprising an antibody or antigen-binding fragment described herein may be suitably buffered, if necessary, and the liquid diluent first rendered isotonic with sufficient saline, polyalcohols, or glucose. For example, one dosage of an antibody or antigen-binding fragment described herein may be dissolved in an isotonic NaCl solution and optionally added to a larger volume of hypodermoclysis fluid prior to being injected at the proposed site of infusion. In some embodiments, the composition comprising an antibody or antigen-binding fragment described herein is provided in a solvent or dispersion medium containing, for example, water, ethanol, polyol (for example, glycerol, propylene glycol, and liquid polyethylene glycol), and suitable mixtures thereof. A composition may also contain adjuvants such as preservatives, wetting agents, emulsifying agents, and dispersing agents. Proper fluidity may be maintained, for example, by the use of a coating, such as lecithin, by the maintenance of the required particle size in the case of dispersion and by the use of surfactants.

In certain circumstances, it will be desirable to deliver an antibody or antigen-binding fragment thereof described herein or suitably formulated pharmaceutical compositions thereof disclosed herein either subcutaneously, intraocularly, intravitreally, parenterally, subcutaneously, intravenously, intracerebro-ventricularly, intramuscularly, intracranially, intrathecally, orally, intraperitoneally, or by oral or nasal inhalation, or by direct injection to one or more cells, tissues, or organs. In some embodiments, an antibody or antigen-binding fragment thereof described herein or composition thereof is injected directly into the cerebrospinal fluid of the subject. In some embodiments, direct injection of an antibody or antigen-binding fragment thereof described herein or composition thereof to human CNS is preferred, for example, delivery is performed concurrently with a surgical procedure or interventional procedure whereby access to the central nervous system tissue is provided. In general, the most appropriate route of administration will depend upon a variety of factors including the nature of the agent (e.g., its stability in the environment of the gastrointestinal tract), and/or the condition of the subject (e.g., whether the subject is able to tolerate oral administration, injection, etc.). In some embodiments, compositions are administered to a subject through only one administration route. In some embodiments, multiple administration routes may be exploited (e.g., serially, or simultaneously) for administration of the composition to a subject.

The administration of therapeutically effective amounts of the compositions of the present disclosure which comprise an antibody or antigen-binding fragment thereof described herein may be achieved by a single administration. For example, a single injection of a sufficient amount of the antibody, antigen-binding fragment thereof or composition thereof may be performed to provide therapeutic benefit to the patient undergoing such treatment. In some circumstances, it may be desirable to provide multiple or successive administrations of the antibody, antigen-binding fragment thereof or composition thereof, either over a relatively short, or a relatively prolonged period of time, as may be determined by the medical practitioner responsible for treatment. In some embodiments, administration of the antibody, antigen-binding fragment thereof or composition thereof to a subject occurs at least one time. In some embodiments, administration of the antibody, antigen-binding fragment thereof or composition thereof to a subject occurs 2, 3, 4, 5, 6, 7, 8, 9, or 10 or more times over the course of treatment.

If desired, the antibody, antigen-binding fragment thereof or composition thereof may be administered in combination with other agents as well, such as, e.g., proteins or polypeptides or various pharmaceutically active agents, including one or more administrations of therapeutic polypeptides, biologically active fragments, or variants thereof. In fact, there is virtually no limit to other components that may also be included, as long as the additional agents do not cause a significant adverse effect upon contact with the target cells or host tissues. The antibody, antigen-binding fragment thereof or composition thereof may thus be delivered along with various other pharmaceutically acceptable agents as required in the particular instance. Such agents may be purified from host cells or other biological sources, or alternatively may be chemically synthesized as described herein.

### Kits

In some aspects, the present disclosure provides a variety of kits comprising antibodies or antigen-binding fragments derived from the approaches described herein or compositions thereof.

The kits comprising an antibody or antigen-binding fragment described herein described herein may be used for a variety of purposes. In some embodiments, kits comprising an antibody or antigen-binding fragment described herein described herein may be used to treat a disease, disorder, or condition.

The kits described herein may include one or more containers housing components for performing the methods described herein, and optionally instructions for use. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

In some embodiments, a kit further comprises a set of instructions for using the compositions comprising an antibody or antigen-binding fragment described herein for carrying out the methods described herein. As used herein, “instructions” can define a component of instruction and/or promotion, and typically involve written instructions on or associated with packaging of this disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral, and electronic communication of any form, associated with this disclosure. Additionally, the kits may include other components depending on the specific application, as described herein.

The kits may have a variety of forms, such as a blister pouch, a shrink-wrapped pouch, a vacuum scalable pouch, a scalable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits, or any of its components, can be sterilized using any appropriate sterilization techniques, such as radiation sterilization, heat sterilization, or other sterilization methods known in the art.

### Applications of Antibodies and Antigen-Binding Fragments Thereof

In some aspects, the present disclosure relates to clinical and commercial applications of antibodies and antigen binding fragments thereof developed through the methods described herein.

In some embodiments, the antibodies and antigen binding fragments thereof developed through the methods described herein may be modified. Examples of modification include the incorporation of one or more non-canonical amino acids into the antibodies and antigen binding fragments thereof developed through the methods described herein. Amino acid modifications may occur at any position within the amino acid chemistry including, but not limited to, the amino acid side chain and the peptide bond between amino acids. In some embodiments, antibodies and antigen binding fragments thereof developed through the methods described herein may be modified with chemistries such as drug molecules. In some embodiments, antibodies and antigen binding fragments thereof may be modified with, for example, one one or more oligonucleotides, cell-penetrating peptides, or small molecules (e.g., cytotoxins, enzyme-inhibitors, etc.).

In some embodiments, the antibodies and antigen-binding fragments thereof developed through the methods described herein may be selected for due to their ability to bind pathogens. In some embodiments, said antibodies and antigen-binding fragments thereof bind to a human pathogen such as, but not limited to, a virus selected from the family of Adenoviridae, Picornaviridae, Herpesviridae, Hepadnaviridae, Coronaviridae, Flaviviridae, Retroviridae, Orthomyxoviridae, Paramyxoviridae, Papovaviridae, Polyomavirus, Poxviridae, Rhabdoviridae, and Togaviridae, a bacteria selected from the group of Mycobacterium tuberculosis, Streptococcus, Pseudomonas, Shigella, Campylobacter, and Salmonella, or a fungus species such as Candida, Aspergillus, Cryptococcus, Histoplasma, Pneumocytis, or Stachybotrus, Bacillus anthracis, Clostridium botulinum, Mycobacterium leprae, Yersinia pestis, Rickettsia prowazekii, Bartonella spp., or another organism such as a parasite that causes malaria, amoebiasis, babesiosis, giardiasis, toxoplasmosis, cryptosporidiosis, trichomoniasis, Chagas disease, leishmaniasis, African trypanosomiasis (sleeping sickness), Acanthamoeba keratitis, and primary amoebic meningoencephalitis (naegleriasis).

In some embodiments, antibodies and antigen-binding fragments thereof developed through the methods described herein may be provided on a vector and expressed in a host cell in order to enable the host cell to attach to a target. In some embodiments, the target may be a human pathogen or a diseased cell or tissue. In some embodiments, the antibodies and antigen-binding fragments thereof are used to generate a T cell expressing a chimeric antigen receptor.

In some embodiments, antibodies and antigen-binding fragments thereof developed through the methods described herein may attached to a particle, microparticle, or nanoparticle. In some embodiments, attachment of said antibodies or antigen-binding fragments thereof to a particle, microparticle, or antigen-binding fragment may be used for diagnostic purposes in a subject. In some embodiments, the particle, microparticle, or nanoparticle conjugated to the antibodies or antigen-binding fragments thereof described herein further comprise an imaging agent for detection either in vivo or ex vivo.

In some embodiments, antibodies and antigen-binding fragments thereof developed through the methods described herein may bind target cells expressing one or more cancer driver genes or genes carrying cancer driver mutations associated with increased risk of cancer. In some embodiments, the cancer driver gene may be, but is not limited to, ATM, ATR, BARD1, BRCA1, BRCA2, BRIP1, CHEK2, CDH1, NFL1, PALB2, PTEN, RAD51C, RAD51D, STK11, TP53, APC, EPCAM, MLH1, MSH2, MSH6, PMS2, MUTYH, P53, P21, NBN, CDKN2A, BAP1, HOXB13, RAS, MAP2K4, FRG1B, SPOP, CASP8, AKT1, KRAS, KDM6A, DNMT3A, U2AF1, FOXA1, FOXA2, KEAP1, RPSAP58, PPP2R1A, PIK3CA, METTL14, SMAD4, CBFB, WT1, CDKN2A, APC, FBXW7, FGFR2, FGFR3, RUNX1, KIT, BRAF, PTEN, CTCF, EGFR, CTNNB1, NFE2L2, IDH2, IDH1, FLT3, PIK3R1, VHL, NRAS, CCND1, MLL2,CDK12, ZFHX3, PTPN11, ATRX, NCOR1, STAG2, MED13, NCOA3, RHEB, ELP2, RBM10, KIAA13241, STXBP5L, LARP1, AB13P, KRAS, CCAR1, CMTR2, ASMTL, SMARCA4, RB1,KEAP1, ARID1A, MUC20, NFE2L2, RYR2, KMT2D, PIK3CA, RASA1, RBL1, FAT, MS4A14, DPPA4, CEP89, NRD1, KLHL4, and PPIP5K2.

### EXAMPLES

### Example 1

Advancements in antibody engineering technologies has lagged far behind the needs for high quality antibodies for research and medicine. Such needs are constantly expanding, fueled by rapid developments of high throughput approaches for genomic profiling and functional screens in recent years that led to the definition of increasing number of biomolecules relevant to diagnosing, monitoring and treating diseases. Animal-dependent methods for generating antibodies are widely used (1) but they are time-consuming and unpredictable (2). In vitro antibody systems based on phage display (3), yeast display (4) or ribosome display (5) could offer greater speed, throughput and reliability. However, despite their advantages, in vitro antibody generation remains under-utilized and further progress in efficiency and throughput are required (6). A strategy to engineer synthetic VHH domain antibodies (nanobodies) against hundreds to thousands of human protein targets simultaneously is described. This strategy combines a recently developed integrated cell-free antibody engineering platform, CeVICA (7), with single-cell sequencing (8) and genome-wide CRISPR/Cas9 knock-out (9) approaches to deliver highly parallel genome-scale antibody generation. The new technologies described herein should catalyze wider adoption of sequence-defined, in vitro engineered antibodies for research and medicine, and enable new capabilities for genome-scale antibody-based technologies.

### Background

Antibodies are essential reagents for research and represent a fast-growing class of therapeutics. However, commercial antibodies are often either polyclonal, and thus not sequence-defined and not renewable, or are monoclonal, but the production of multiple monoclonal antibodies is expensive and time-consuming (1) (2). A method for rapid and cost-effective generation of sequence-defined antibodies would enable the creation of new antibody-based tools.

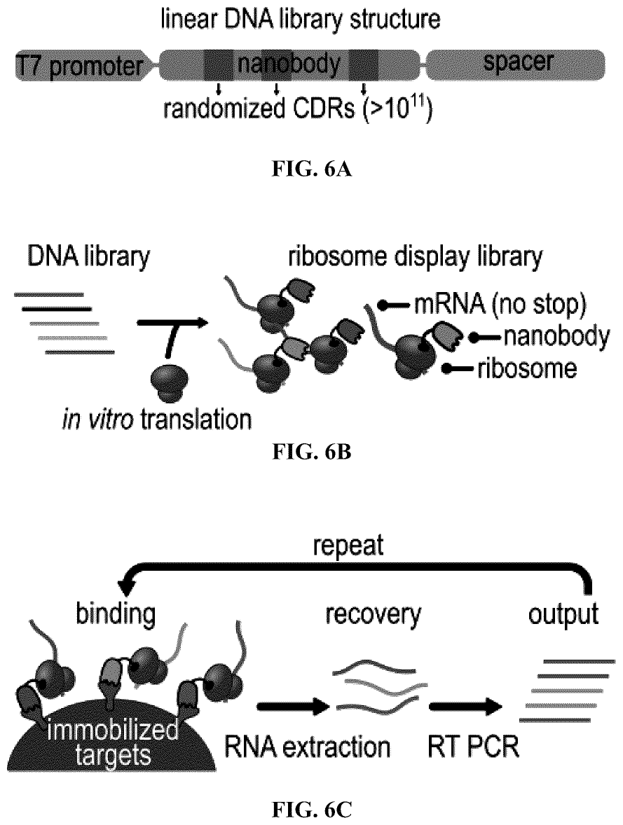

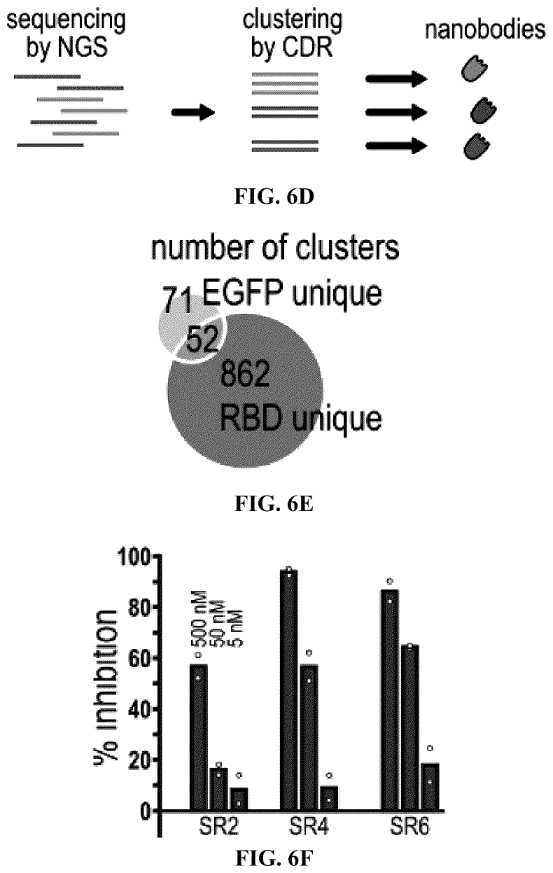

A cell-free method for generating and screening massive antibody libraries was recently developed, consisting of a fully synthetic library with up to 10¹⁰ or 10¹¹ independent single-chain VHH nanobodies, which was described in PCT Application No. PCT/US2021/051925, which claims priority to U.S. Application Nos. 63/083,073 and 63/221,663, all three of which are incorporated by reference herein in their entirety. This method is shown in FIG. 6. For example, a linear DNA library structure is provided (FIG. 6A), wherein a nanobody sequence with randomized CDRs is controlled by a T7 promotor and is found upstream of a spacer sequence. Using ribosome display technology to produce RNA-tagged nanobodies from the random VHH (FIG. 6B), nanobodies that bound immobilized targets were selected, and high affinity neutralizing nanobodies were identified. For example, nanobodies against SARS-COV-2 spike receptor binding domain (RBD) and EGFP were successfully selected using three rounds of selection (FIG. 6C). In this example, the output was analyzed by next generation sequencing (NGS) and computational clustering by CDR sequences (FIG. 6D), which led to more than 800 distinct binding clusters. In this case, each cluster represented a predicted binder of RBD (FIG. 6E). Testing and affinity maturation of a subset of these clusters yielded potent SARS-COV-2 neutralizing nanobodies (FIG. 6F).

This small single-chain VHH domain antibody format offers good physiochemical properties and amenability to engineering. By using the cell-free display approach and ribosome display, CeVICA can screen extremely high diversity input libraries rapidly. Computational clustering of identified binder prediction enables more efficient iterative screens and optimization. The system also has the advantage that small (14 kD) single-chain nanobodies are identified that have useful properties for screening (e.g., shorter sequences) and downstream applications because, for example, they are stable with Tₘ up to 90° C.; they are feasible to scale up for production; and they are smaller to penetrate tissues. Prior methods using phage and yeast display had lower diversity (for example, around 10⁹) and slower cycles (due to the time needed to culture cells). The use of CeVICA has previously been shown to successfully engineer SARS-CoV-2 neutralizing antibodies (7).

The advantages of CeVICA can be leveraged as explained herein to develop novel integrated pipelines for simultaneous generation of antibodies against hundreds to thousands of human proteins. The comprehensive analysis of all binders in the output library permits flexible integration with high throughput sequencing technologies such as droplet-based single cell sequencing and additional computational tools that could enable greater throughput. This system is well suited for analyzing cell surface proteins, due to their critical role in cell signaling and as important therapeutic targets (10), while being versatile enough for other antigen classes such as intracellular proteins, peptides protein complexes, and peptide-MHC complexes (PMID: 9391117, PMID: 14523945, PMID: 33649166). These goals will be achieved through the following two complementary approaches.

### Antibody Generation for Diverse Antigen Classes Using Single Cell Open Reading Frame (ORF) Expression and Sequencing

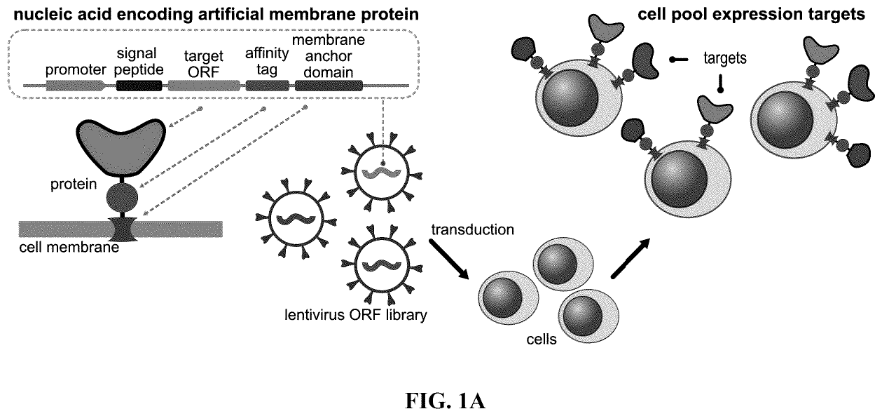

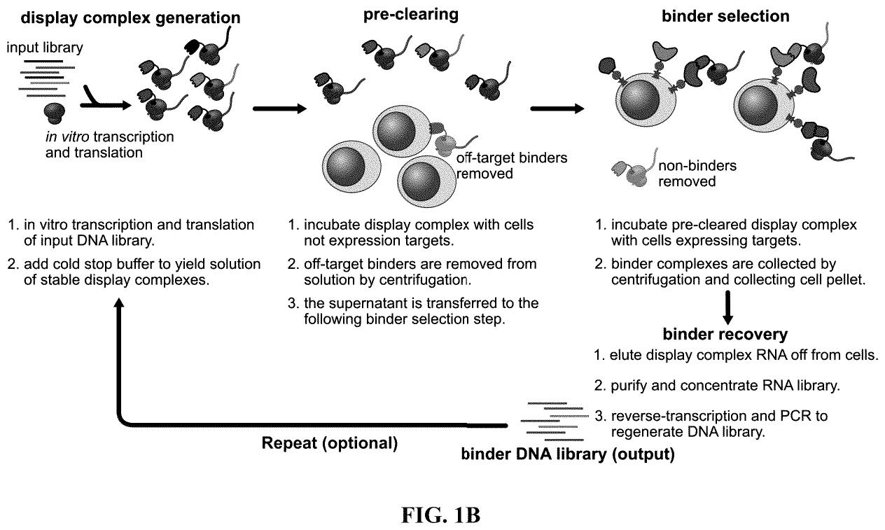

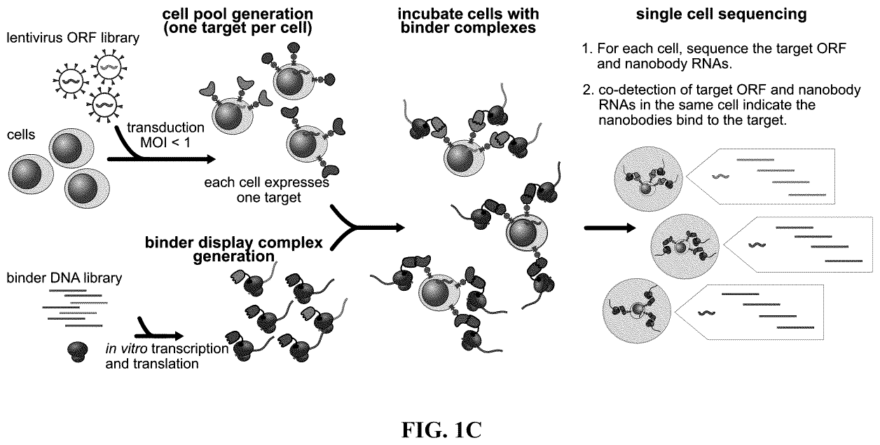

This approach will create a generalizable pipeline for generating antibodies for selected targets at the scale of hundreds. For example, around 200 targets of interest can be selected, and a two-stage process can be used to first select antibodies for the targets as a pool, then map each antibody to their targets (FIGS. 1A-1C). It is anticipated that this two-stage design will minimize background noise during antibody-antigen mapping. Finally, antibodies will be validated by independent biochemical assays.

### Establish a Universal Strategy to Ectopically Express Target Proteins on CHO Cell Surface.

Around 200 target proteins or protein domains can be selected that include diverse classes such as, but not limited to, membrane proteins/domains (e.g., CD39, Tim3, CD28, CD4), intracellular proteins (e.g., STING), short peptides (as peptide-carrier protein fusion) and peptide-MHC I complexes (as a single-chain format (11)). Multi-pass transmembrane domains or domains that are not stable alone are usually excluded. Selected targets are cloned into lentiviral vectors to allow expression as artificial cell membrane proteins by cloning in-frame, a N-terminal signal peptide, a C-terminal single pass transmembrane domain or a Glycosylphosphatidylinositol (GPI) anchor and a 3xFlag affinity tag (see FIG. 1A). These constructs are assayed to confirm successful expression of the targets on the cell surface by immunostaining. In the event of suboptimal cell surface expression, different signal peptide and transmembrane domain/GPI anchor combinations can be assayed to find the most optimal format. These lentiviral constructs can also contain unique barcodes in the ORF's 3′UTR to allow reading the identity of the ORF in single cell sequencing workflows. Once complete, the ORF lentivirus constructs are pooled together and an ORF lentivirus pool is generated.

### Pooled Selection and Antibody-Antigen Mapping

Targets are produced as a pool by cells (for example CHO cells) transduced by the ORF lentivirus pool, and the target proteins can be immobilized on magnetic beads via the 3xFlag tag. Pooled antibody selection is performed using CeVICA under selection conditions optimized for high affinity binders. For example, as shown in FIG. 1B, a display complex is generated by in vitro transcription and translation of an input DNA library, followed by cold stop buffer to yield a solution of stable display complexes. These display complexes can be pre-cleared to remove off-target binders by incubating the display complexes with cells not expressing the target. The off-target binders are removed from the solution by centrifugation. The supernatant is then transferred for binder selection. Binder selection occurs by incubating the display complexes (which may be pre-cleared) with cells expressing targets, and with the removal of non-binders. The binder complexes that are bound are then collected by centrifugation, and the cell pellets collected. Binder recovery occurs by eluting the display complex RNA from the cells, purifying and concentrating the RNA library, and regenerating the DNA library by reverse-transcription and PCR. The output binder DNA library can optionally be used in a repeat process to enrich for binder complexes that bind the cells.

Successful antibody selection is confirmed by monitoring the recovered RNA yield at each selection cycle as well as enrichment of full-length antibody sequences in the output library. These metrics were shown to be early indications of successful selection (7). The selection output is used to generate computationally predicted antibody clusters and serve as a database for antibody-antigen mapping. The mapping is performed by first displaying the selection output library using ribosome display, then incubating this display complex pool with CHO cells infected by targets ORF lentivirus library at low MOI (such that most cells will only express a single ORF target) (FIG. 1C). These incubated cells are then processed by 10× 3′ expression kits to read the target expressed by each cell and the antibody sequences attached to that cell. A computational pipeline is used on these sequencing data to establish the link between antibodies and their targets. The SARS-COV-2 receptor binding domain (RBD) can be included as one of the targets to serve as an internal control and spike in previously identified RBD VHH antibody sequences (7) in the initial library. This known antibody-antigen pair will serve as a positive control for the mapping process and aid in determination of optimal parameters for the computational pipeline.

### Antibody Validation

Independent binding assays can be used to validate identified antibodies. Selected antibody sequences are synthesized, cloned and expressed in E. Coli. Three assays are well suited for use in validating binding and specificity of each antibody: 1) an automated ELISA workflow to measure both target binding and off-target binding in 96 well plates; 2)., flow cytometry for targets with existing antibodies to confirm quantitative correlation between the newly selected antibodies and existing antibodies; and 3) Bio-Layer Interferometry (BLI) to quantitatively assay antibody binding kinetics.

Simultaneous GENERATION of antibodies Against All Cell Surface Proteins Using Single Cell Sequencing and Cas9/gRNA Knock-Out (KO)

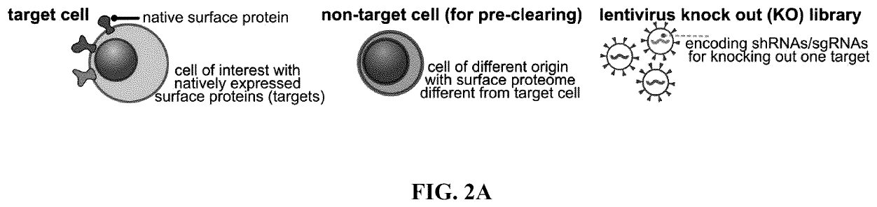

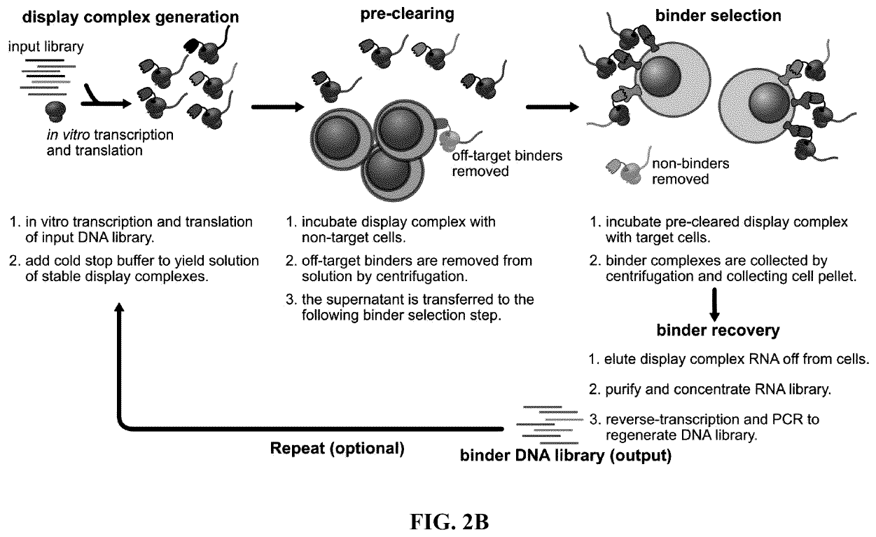

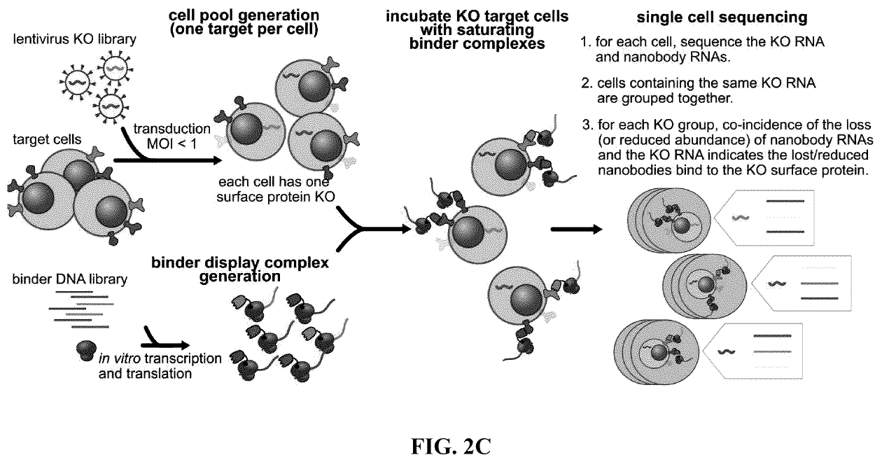

A pipeline is established to generate antibodies against all cell surface proteins expressed by a group of cells. This pipeline adopts a two-stage process (FIGS. 2B-2C). First, display complexes that bind to cells of interested are enriched in a method similar to that as described above. However, in this approach, ectopic expression of targets is not required, because the targets are cell surface proteins natively expressed by the chosen cells. Second, the expression of proteins on the target cells are either reduced or eliminated in a KO step to select for binder complexes that no longer bind to the target cells. This approach is achieved via the following steps.

### Determine Optimal Conditions and Parameters for Detecting Nanobodies that Bind Surface Proteins

Pilot studies using a nanobody library that includes hundreds of sequences that encode spike RBD-binding and EGFP-binding nanobodies, prepared as described above, can be performed prior to screening against endogenous surface proteins. In such pilot studies, the target cells can be the Jurkat T cell line (an easy to grow suspension line) expressing RBD on the surface. The lentivirus-encoded sgRNAs are introduced to delete RBD in varying fractions of cells and monitor sgRNA-mediated loss of nanobody binding with scRNA-seq of nanobody RNAs and sgRNA barcodes. The loss of binding of our established nanobodies in cells lacking RBD or EGFP on the surface should be observed (measured as a reduction in abundance of linked nanobody RNA sequences). These positive control screens can be used to tune experimental protocols (such as binding conditions to reduce non-specific binding, fraction and number of cells expressing the sgRNA needed to observe dropout of nanobody binding, sequencing depth needed for sufficient recovery of nanobody clusters) and computational pipelines (clustering algorithms based on biophysical parameters to increase power and speed, cut-off parameters for minimal size of clusters and their size fold change to maximize signal to noise ratio), with the goal of maximizing identification of positive controls across a spectrum of binding affinities and off-rates.

### Characterize Human Cell Lines Suitable for Genome Wide Surface Protein Antibody Generation

Target cells of interest are identified that natively express target proteins, such as native surface proteins (FIG. 2A). For example, three cell lines can be chosen, such as HEK293T sus, Jurkat and SH-SY5Y (target cells). These cell lines have distinct tissue of origins such that their collective cell surface expressed proteins represent a larger proportion of all surface proteins in the human genome. They are also known to be compatible with CRISPR/Cas9 and droplet-based single cell sequencing. RNA-seq can be performed on the cell lines to confirm their surface protein expression profile and to construct knock-out (KO) libraries. For example, a lentivirus knockout library can be constructed that contains shRNAs or sgRNAs for knocking out one target (FIG. 2A). A Cas9/gRNA lentiviral library (using constructs based on the CROP-seq method (12)) targeting the entire human surface proteome can also be prepared (10). The efficacy of Cas9/gRNA KO system for several well characterized cell surface proteins (such as CD28, HLA) can be assayed by immunostaining. Finally, pre-clearing cells of different origin and different surface proteome as the cell of interest can be used (FIG. 2A).

### Pooled Selection and Antibody-Antigen Mapping

In this approach, live target cells in single cell suspension serve as the solid phase with immobilized targets for pooled selection of antibodies (FIG. 2A). The same steps as in the above application are used to generate a pool of all predicted antibody clusters to be used for antibody-antigen mapping (FIG. 2B). For example, display complexes are generated by in vitro transcription and translation of an input DNA library. Starting input library sizes of up to 10¹² can be used by optimizing our existing protocol to incorporate more input DNA library amount in the initial selection cycle (which can be achieved by using larger reaction volumes). This is followed by cold stop buffer to yield a solution of stable display complexes. The display complexes can be pre-cleared by incubating them with pre-clearing cells. Cells from other species and/or cell types (e.g., hamster CHO cells, Drosophila S2 cells) can be used for the pre-clearing, to provide for pre-clearing of non-specific binders, which can be, for example, lipid/sugar binders and promiscuous binders. The nanobodies that do not bind non-specifically to cells are retained. The display complexes that bind off-target binders are removed from the solution, for example by centrifugation. The supernatant is then used in the binder selection step. The binder selection step enriches for surface binders, thereby reducing the complexity of the input library for mapping. For example, the pre-cleared library can be incubated with intact Jurkat cells and positively selected for binding nanobodies. Binder selection occurs by incubating pre-cleared display complexes with target cells. The non-binders are removed, and binder complexes that bound to the target cells are collected, for example by centrifugation where the cell pellets are collected. The binders are recovered by eluting the display complex RNA from the cells, purifying and concentrating the RNA library. The RNA library is then reverse transcribed, and PCR is used to regenerate the DNA library. These steps can be repeated on the output binder DNA library to enrich the library. The library can be monitored through multiple cycles of selection by measuring recovered RNA yield and enrichment for full-length nanobody sequences at each selection cycle, which can be predictive of successful positive selection (7). The resulting output library will thus be enriched for binders of Jurkat cells and depleted for non-specific cell binders.

Mapping is performed by first displaying the selection output library using ribosome display, then incubating this display complex pool with target cells in which expression of the protein to be mapped is reduced or eliminated. For example, CRISPR can be used to deplete specific surface proteins in individual cells and identify nanobodies that do not bind cells lacking each protein (FIG. 2C). Alternatively, CRISPRi (PMID: 23849981) or RNA interference (PMID: 21953743) can be used to reduce the level of surface proteins for mapping of nanobodies targeting these surface proteins. Specifically, target cells are infected with a Cas9/gRNA lentivirus KO library at low MOI, such as MOI<1. This will generate a cell pool with approximately one target per cell, where ideally each cell will have one surface protein KO. A binder DNA library is transcribed and translated in vitro to generate binder display complexes. The KO target cells are incubated with saturating binder complexes. These incubated cells are then processed, for example with 10× 3′ expression kits, to read the gRNA identity of each cell and the antibody/nanobody sequences of those binder complexes that attached to that cell. For each cell, the sequence of the KO RNA and the nanobody RNA are sequenced. The cells containing the same KO RNA are grouped together.

For each KO group, the co-incidence of the loss (or reduced abundance) of nanobody RNAs and the KO RNA indicates that the lost/reduced nanobodies bind to the KO surface protein. For example, a computational pipeline is used to analyze the sequencing data, in which all antibody sequences bound to cells with the same gene KO are combined and antibody clusters are generated for each gene KO. This list of antibody clusters is then compared to the selection output antibody clusters (the list prior to antibody-antigen mapping) to identify antibody clusters that are lost or reduced in size for each gene KO. These lost antibodies associated with each gene KO indicates potential binding of the antibody to the gene's encoded protein. Previously identified VHH sequence (13), such as for anti-PD-L1, can be spiked in the initial library and used as the positive control for optimization of experimental protocols and computational pipeline.

### Antibody Binding and Specificity Validation Using Computational Algorithm.

The whole surface proteome antibody screen provides a rich dataset from which antibody binding and specificity properties can be quantitatively derived. Biochemical assays, such as ELISA and BLI, can be used to characterize binding properties of a subset (for example, about 100) of identified antibodies to serve as an independent quantification dataset. The degree of loss (antibody cluster size reduction) for each gene KO is used to establish a quantitative model to predict binding strength by correlating each antibody's degree of loss with their binding properties measured by, for example, ELISA and BLI. Further, the degree of loss patterns for each antibody cluster is compared across all surface protein KOs to construct a map of specificity for each antibody. Antibodies with high degree of loss to only one surface protein KO but very low degree of loss to all other proteins KO indicates high specificity. The analysis takes into consideration that KO of one gene may interfere with the expression of other genes. These computational algorithms can provide a powerful approach to rapidly identify high quality antibodies across the surface proteome with minimal laborious experimental testing.

If the signal is not high enough using the proposed dropout screen, a positive binding mapping approach can be used. For example, epitope-tagged target proteins (or their domains) are ectopically expressed on the cell surface using an N-terminal signal peptide, C-terminal single pass transmembrane domain or GPI anchor. A pipeline similar to the above approach Aim 2 can be used to perform single cell sequencing and read out ORF barcodes and bound nanobody sequences per cell, and thus identify nanobody-target pairs. As this approach becomes more feasible through gene synthesis, synthetic gene libraries can be synthesized and expressed in cell lines of non-mammalian species such as Drosophila to reduce competition with the corresponding endogenous proteins. It is expected that thousands of cell-line specific nanobodies can be identified and many mapped to their targets with high specificity. To address the possibility that KO of a single protein may lead to loss or other proteins (e.g. complexes that depend on each component for stability or other dependencies such as trafficking or signaling) and confound mapping, single in vitro recombinant proteins can be generated and immobilized on magnetic beads for definitive mapping. For KOs that cause cell death, CRISPRi (PMID: 23849981) partial knock down or RNA interference (PMID: 21953743) will be used to allow cells to survive.

Using Ribosome Display Complex Pools as Highly Multiplex Protein Profiling Reagents

Another example of the use of the large collection of antibodies is antibody-based cell surface proteome profiling, in a process that is highly similar to CITE-seq which uses oligo conjugated antibodies. For example, a barcoded antibody pool can be generated using a nanobody DNA library that has been transcribed and translated in vitro. The result is a barcoded nanobody pool. This pool can be used to used to bind to target cells of interest, followed by high-throughput sequencing to quantify the nanobody RNA sequences and barcodes of the binding targets that bind to the target cell. An example of scheme for sequencing using Next-Generation Sequencing (NGS) of the library for nanobody-target mapping is found at FIG. 3.

### Using Whole Cell to Present Antigen on the Cell Surface for Nanobody Selection

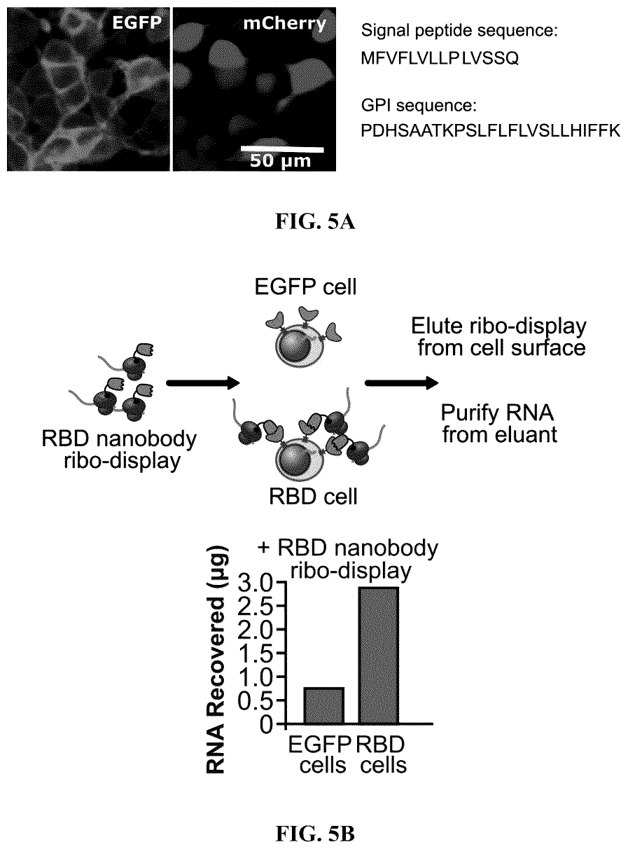

As another example, whole cells can be used to present antigen for on the cell surface for nanobody selection. As an example, EGFP protein was expressed as an artificial cell surface protein by adding an N-terminal signal peptide and a C-terminal GPI anchor sequence (FIG. 5A). Whole cell can act as solid surface presenting antigen for selection of nanobody ribosome display complex, the receptor binding domain (RBD) of SARS-COV-2 spike protein and RBD nanobodies are used to test feasibility, EGFP can be used as negative control. For example, RBD nanobody ribo-display, as described above, was reacted with both he EGFP cells, and cells expressing RBD, and the ribo-displays that bind to the cell surface of either cell was clute (FIG. 5B). RNA from the eluant was purified and quantitated, the amount of RNA recovered, in μg, was determined.

Together, these approaches provide for high throughput antibody generation for diverse antigen types at a range of scales. These approaches allow highly parallel antibody engineering at low cost, create a large collection of antibodies that enable antibody-based cell surface proteome profiling (e.g., using CITE-seq), offer the potential to generate antibodies against “difficult” targets (e.g., glycan moieties and other post-translational modifications including, but not limited to, hydroxylation, farnesylation, isofarnesylation, lipidation, addition of a linker for conjugation or functionalization, phosphorylation, de-phosphorylation, acetylation, de-acetylation, SUMOylation, glycosylation, nitrosylation, methylation, and ubiquitination), and pave the path towards antibody generation for all proteins in the human genome using the Cas9/gRNA based strategy.

### REFERENCES

1. A. Gray, A R. M. Bradbury, A. Knappik, A. Plückthun, C. A. K. Borrebaeck, S. Dübel, Animal-free alternatives and the antibody iceberg. Nat. Biotechnol. 38, 1234-1239 (2020).
2. A. C. Gray, A. R. M. Bradbury, A. Knappik, A. Plückthun, C. A. K. Borrebaeck, S. Dübel, Animal-derived-antibody generation faces strict reform in accordance with European Union policy on animal use. Nat. Methods. 17, 755-756 (2020).
3. J. Huo, A. Le Bas, R. R. Ruza, H. M. E. Duyvesteyn, H. Mikolajek, T. Malinauskas, T. K. Tan, P. Rijal, M. Dumoux, P. N. Ward, J. Ren, D. Zhou, P. J. Harrison, M. Weckener, D. K. Clare, V. K. Vogirala, J. Radecke, L. Moynié, Y. Zhao, J. Gilbert-Jaramillo, M. L. Knight, J. A. Trec, K. R. Buttigieg, N. Coombes, M. J. Elmore, M. W. Carroll, L. Carrique, P. N. M. Shah, W. James, A. R. Townsend, D. I. Stuart, R. J. Owens, J. H. Naismith, Neutralizing nanobodies bind SARS-COV-2 spike RBD and block interaction with ACE2. Nat. Struct. Mol. Biol. (2020), doi: 10.1038/s41594-020-0469-6.
4. E. T. Boder, K. D. Wittrup, Yeast surface display for screening combinatorial polypeptide libraries. Nat. Biotechnol. 15, 553-557 (1997).
5. J. Hanes, A. Plückthun, In vitro selection and evolution of functional proteins by using ribosome display. Proc. Natl. Acad. Sci. U. S. A. 94, 4937-4942 (1997).
6. A. R. M. Bradbury, S. Sidhu, S. Dübel, J. McCafferty, Beyond natural antibodies: The power of in vitro display technologies. Nat. Biotechnol. 29, 245-254 (2011).
7. Chen, X., Gentili, M., Hacohen, N. et al. A cell-free nanobody engineering platform rapidly generates SARS-COV-2 neutralizing nanobodies. Nat. Commun. 12, 5506 (2021). https://doi.org/10.1038/s41467-021-25777-z.
8. E. Z. Macosko, A. Basu, R. Satija, J. Nemesh, K. Shekhar, M. Goldman, I. Tirosh, A. R. Bialas, N. Kamitaki, E. M. Martersteck, J. J. Trombetta, D. A. Weitz, J. R. Sanes, A. K. Shalck, A. Regev, S. A. McCarroll, Highly parallel genome-wide expression profiling of individual cells using nanoliter droplets. Cell. 161, 1202-1214 (2015).
9. O. Shalem, N. E. Sanjana, E. Hartenian, X. Shi, D. A. Scott, T. S. Mikkelsen, D. Heckl, B. L. Ebert, D. E. Root, J. G. Doench, F. Zhang, Genome-Scale CRISPR-Cas9 Knockout Screening in Human Cells. Science. 343, 84-87 (2014).
10. D. Bausch-Fluck, U. Goldmann, S. Müller, M. van Oostrum, M. Müller, O. T. Schubert, B. Wollscheid, The in silico human surfacecome. Proc. Natl. Acad. Sci. U. S. A. 115, E10988-E10997 (2018).
11. Y. Y. L. Yu, N. Netuschil, L. Lybarger, J. M. Connolly, T. H. Hansen, Cutting Edge: Single-Chain Trimers of MHC Class I Molecules Form Stable Structures That Potently Stimulate Antigen-Specific T Cells and B Cells. J. Immunol. 168, 3145-3149 (2002).
12. P. Datlinger, A. F. Rendeiro, C. Schmidl, T. Krausgruber, P. Traxler, J. Klughammer, L. C. Schuster, A. Kuchler, D. Alpar, C. Bock, Pooled CRISPR screening with single-cell transcriptome readout. Nat. Methods. 14, 297-301 (2017).
13. J. R. Ingram, M. Dougan, M. Rashidian, M. Knoll, E. J. Keliher, S. Garrett, S. Garforth, O. S. Blomberg, C. Espinosa, A. Bhan, S. C. Almo, R. Weissleder, H. Lodish, S. K. Dougan, H. L. Plocgh, PD-L1 is an activation-independent marker of brown adipocytes. Nat. Commun. 8 (2017), doi: 10.1038/s41467-017-00799-8.
14. Sarkizova S, Klaeger S, Le P M, Li L W, Oliveira G, Keshishian H, Hartigan C R, Zhang W, Braun D A, Ligon K L, Bachireddy P, Zervantonakis I K, Rosenbluth J M, Ouspenskaia T, Law T, Justesen S, Stevens J, Lane W J, Eisenhaure T, Lan Zhang G, Clauser K R, Hacohen N, Carr S A, Wu C J, Keskin D B. A large peptidome dataset improves HLA class I epitope prediction across most of the human population. Nat Biotechnol. 2020 February;38 (2): 199-209. doi: 10.1038/s41587-019-0322-9.

### Example 2

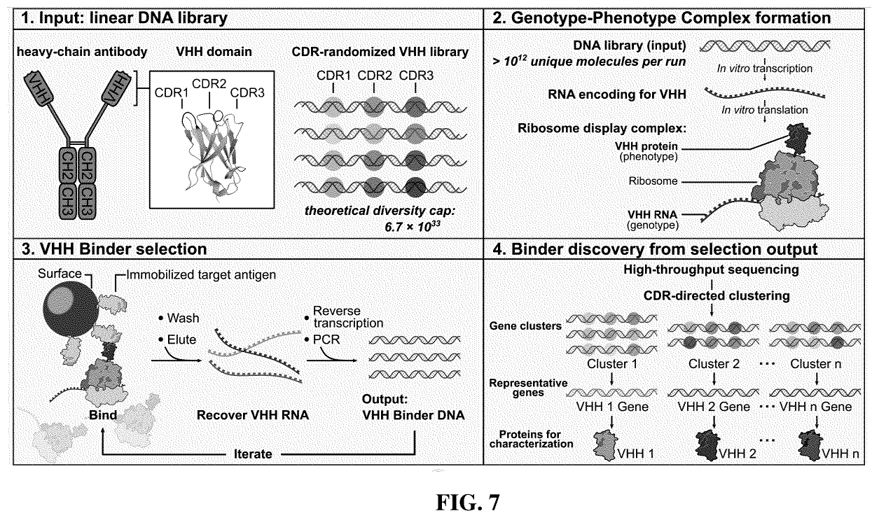

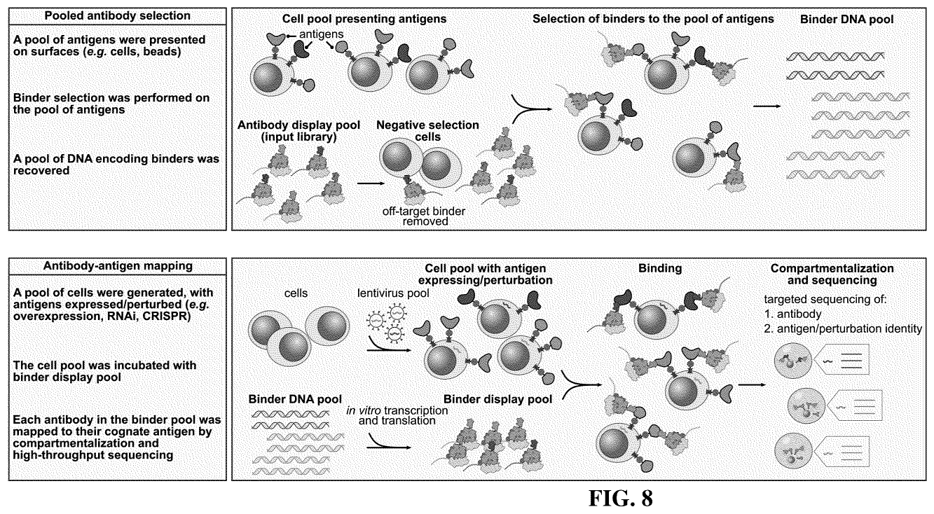

The above-referenced CeVICA platform, which is an integrated VHH antibody discovery platform that rapidly and reliably generated hundreds of VHHs against one antigen, was used to generate VHHs against the receptor binding domain of SARS-COV-2 spike (RBD) and optimized one VHH to become a potent virus neutralization agent (FIGS. 6, 7). A platform was developed that can perform antibody selection against multiple antigens simultaneously in one mixed pool. This platform is composed of two stages: First, pooled antibody selection yields a large pool of antibodies with each antibody likely binding to at least one antigen in a pool of antigens. Second, antibody-antigen mapping finds the cognate antigen of each of the antibodies discovered by the pooled antibody selection in high-throughput (see FIG. 8). This platform is adaptable with different antibody formats, such as VHH and scFV, and different types of antigens, such as folded protein domains, short peptides, membrane proteins, protein-protein or protein-peptide complexes, and post-translational modifications. This Examples provides a proof-of-principle by generating and mapping VHH against a collection of 27 antigens.

To prepare antigens for VHH selection, the 27 antigens were overexpressed in HEK293T cells as cell surface membrane proteins, among which 20 protein domains from CD4, CD3E, CD28, CD19, CD25, CD45, PD1, PDL1, CTLA4, TIM3, UBE3A, ILIR2, CD14, CD177, IL7R, IL2, AR, CDH2, HER2, STING1 were expressed on the cell surface by adding a N-terminal signal peptide and C-terminal GPI anchor, 7 GPCRs were expressed as full-length protein or with an additional N-terminal signal peptide. RBD and EGFP were expressed on the cell surface by adding a N-terminal signal peptide and C-terminal GPI anchor and used as controls (Tables 2 and 3).

Whether ribosome display complex is compatible with compartmentalization (i.e., separation of a solution of a larger volume into compartments of smaller volumes such as generating a solution comprising compartments like water-in-oil droplets via microfluidics or dispensing a solution into compartments like microwells on a plate). and sequencing by a commercially available single cell sequencing system from 10x Genomics was tested. A ribosome display pool was generated using a mixture of four VHH-spacer DNA, among which two VHH, SR6v7 and SR1 (see PMID: 34535642), binds RBD. with SR6v7 having a higher affinity than SR1. The other two VHH, GP6 (see PMID: 34535642), GP3k1k (see PMID: 20010839), bind EGFP. This four VHH ribosome display pool was incubated with a pool of cells expressing RBD and cells expressing EGFP on the surface. The VHH RNA associated on the cells and the antigen transcripts expressed by the cells were quantified by the 10x Genomics droplet system using reverse transcription, PCR, and illumina sequencing. Quantification of

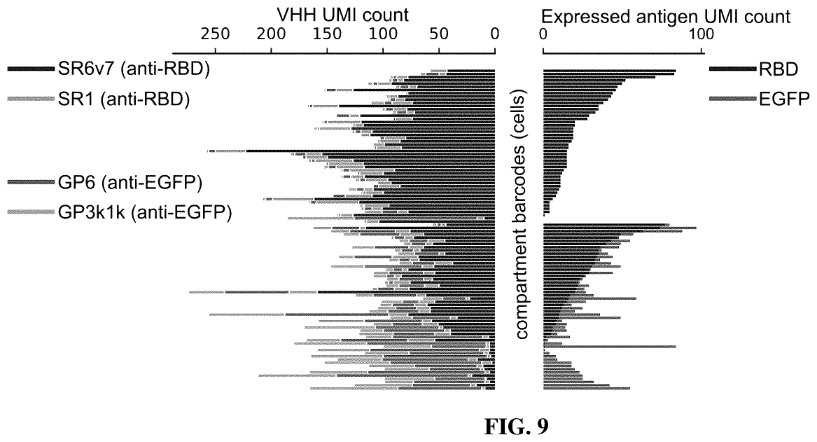

VHH and antigen transcripts in a droplet was based on UMI count of VHH reads and antigen transcript reads that contain the 10× Barcode which uniquely labels the droplet compartment (compartment barcodes). Mostly anti-RBD VHHs in compartments containing only RBD cells were observed (FIG. 9, top), and mostly anti-EGFP VHHs in compartments containing only EGFP cells were observed (FIG. 9, bottom). Some compartments showed both EGFP and RBD transcripts (FIG. 9, middle), which could have resulted from multiple cells entering one compartment, and these compartments showing a more even mixture of anti-RBD and anti-EGFP VHHs compared to the compartments containing only one antigen transcript. In addition, the higher affinity anti-RBD VHH, SR6v7, was consistently detected at higher quantities than SR1 in all compartments, indicating that the quantification workflow faithfully reports affinity related information. Overall, these results show ribosome display complex is fully compatible with compartmentalization and sequencing.

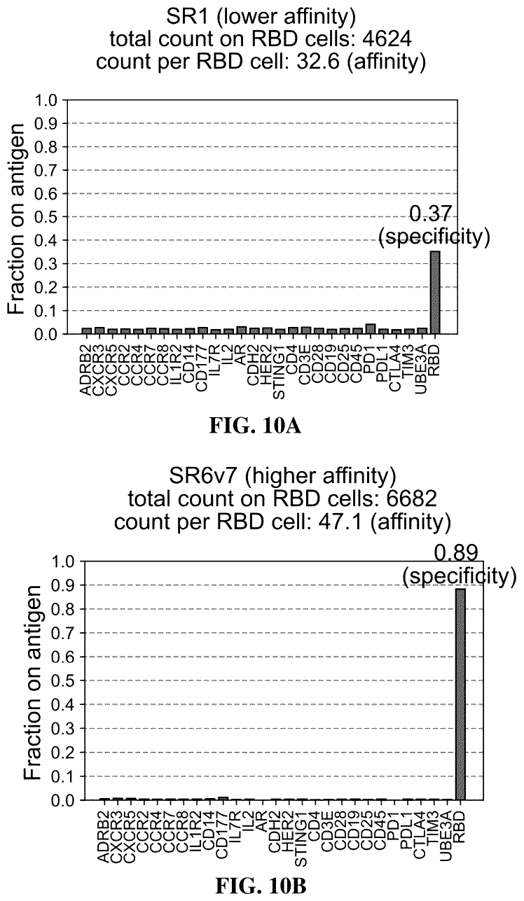

Performed pooled VHH selection on a pool of cells expressing the 27 antigens on the surface, and an E. coli ribosomal RNA band was observed in recovered RNA from the fifth iteration, a strong indication that VHH selection was successful. To perform VHH-antigen mapping, the output DNA library from the fifth iteration was mixed with SR6v7 and SR1 anti-RBD positive control VHH-spacer DNA (see Materials and Methods, below), and this DNA mixture was used for generating ribosome display pool. The ribosome display pool was then incubated with a pool of cells with each one expressing one of the 27 antigens plus RBD expressing cells (as a positive control). The cells in solution were then compartmentalized, sequenced and analyzed (see Materials and Methods, below). The distribution of the positive control VHH over the 28 antigens was checked, and it was found that the two anti-RBD VHH were mainly present on RBD cells (FIGS. 10A and 10B), with the higher affinity SR6v7 having 89% on RBD cells (FIG. 10B), while SR1 having 37% on RBD cells (FIG. 10A). Both VHH showed some presence on other antigen expressing cells, and these lower levels may be due to non-specific binding or background signal. This analysis generated values that correlate with VHH-antibody affinity (count per cell) and specificity (fraction of VHH on its cognate antigen expressing cells).

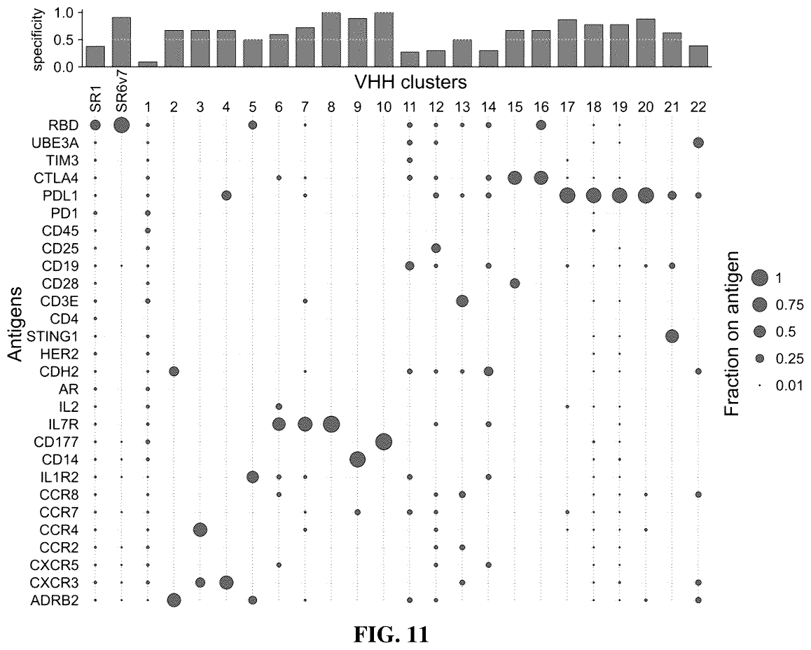

The distribution of other VHHs from the pooled VHH selection output was also evaluated. All VHHs were clustered based on their CDR sequence similarity and calculated their cell number normalized fraction on antigen (see Materials and Methods, below), and thousands of clusters were found that showed similar distribution characteristics as the two positive control anti-RBD VHHs but towards a different antigen. Among the 22 example VHH clusters (FIG. 11), some showed higher specificity, such as clusters 8 and 20, while some others showed lower specificity, such as clusters 5 and 22. An example of off-target binder cluster, cluster 1, was also shown which binds cells expressing different antigens at similar levels, and its true cognate antigen may be a natively expressed surface protein by HEK293T cells.

These results demonstrated the feasibility of parallel antibody discovery and validation in vitro at high throughput by combining in vitro display and single cell sequencing. Our technology platform described in this study could power large scale antibody discovery and validation efforts, leading to significant acceleration and cost-reduction in the development of antibody dependent applications.

### Materials and Methods

### Constructs and DNA

DNA encoding antigens (amino acid sequences were shown in Table 1) were obtained by gene synthesis (Integrated DNA Technologies) and cloned into pTRIP vector with C-terminal DNA barcode sequence unique for each antigen and the binding sequence of 10× Genomics Capture Sequence 2 (5′-3′: GCTCACCTATTAGCGGCTAAGG) by digestion with restriction enzyme and ligation with T4 DNA ligase. DNA encoding VHH (SR6v7: anti-RBD, SR1: anti-RBD, GP6: anti-EGFP, GP3k1k: anti-EGFP) in frame with C-terminal spacer for ribosome display (see PMID: 34535642) containing the 10x Genomics Capture sequence 1 (5′-3′: GCTTTAAGGCCGGTCCTAGCAA) were obtained by gene synthesis and cloned into pBx1vector (pBx1-T7 promoter-VHH-spacer, amino acid sequences of VHHs are shown in Table 4) by Gibson assembly (NEBuilder® HiFi DNA Assembly Master Mix, New England Biolabs). The pBx1-T7 promoter-VHH-Spacer plasmids were used as template in PCR reactions with DNA recovery primer pair (see PMID: 34535642) to generate positive control VHH-spacer DNA, the PCR reactions were purified using NucleoSpin Gel and PCR Clean-Up Kit (Takara). Cell culture

HEK293T cells were cultured in DMEM, 10% FBS (ThermoFisher Scientific), PenStrep (ThermoFisher Scientific) at 37° C. with 5% CO₂. To produce lentivirus, HEK293T cells were seeded at 0.8×10⁶ cells per well in a 6 well plate and were transfected the same day with TransIT-293 Transfection Reagent and a mix of DNA containing 1 μg pTRIP vector expressing antigen(s), 0.7 μg psPAX2 and 0.3 μg pCMV-VSV-G. Lentiviral particles were collected 48 hours post transfection by collecting culture media from transfected well, then centrifuged at 4000 rpm for 5 minutes at room temperature, after which supernatants containing lentiviral particles were collected. To generate stable cell line by lentivirus transduction, HEK293T cells were seeded at 0.8×10⁶ cells per well in a 6 well plate and transduced by adding 50 μl virus solution to the well on the same day, 48 hours after transduction the culture media were discarded and transduced cells were selected in culture media containing 2 μg/ml puromycin for 48 hours, after which cells were maintained in culture media without puromycin.

### VHH Selection Against Whole Cells

Whole cells for VHH selection were prepared by removing culture media from cell, then resuspending cells in PBS, then centrifuging at 400g for 2 minutes at 4° C. VHH selection were performed by iterations of first preparing input library ribosome display pool as described previously (see PMID: 34535642), then incubating ribosome display pool solution with cells not expressing target antigens, then transfer supernatant to cells expressing target antigens, ribosome display complexes bound to cells expressing target antigens were recovered by incubating cells in PBS solution containing 5 mM EDTA and 0.1 μg/μl Bovine Serum Albumin for 5 minutes, RNA were extracted from the eluent using Monarch RNA Cleanup Kit (New England Biolabs).

### VHH-Antigen Mapping: Compartmentalization

Ribosome display pool solution was incubated with cells expressing antigen on their cell surface for 1 hour at 4° C., the cells were washed three times and resuspended at a concentration of 400 cells/μl. 43.2 μl of the cell suspension was loaded on to Chromium Next GEM Chip G (10x Genomics) according to the Chromium Next GEM Single Cell 3′ Reagent Kits v3.1 (10x Genomics) manual and cells with bound ribosome display complexes were compartmentalized in water-in-oil droplets using the Chromium Controller (10x Genomics) followed by reverse transcription. The reverse transcription cleanup product containing cDNA of VHH RNA and antigen RNA transcripts was used as template in PCR to selectively amplify a region of VHH cDNA covering all three CDRs and a region of antigen cDNA covering the DNA barcode sequence. The PCR products were purified and quantified for sequencing by illumina Miseq.

To test anti-RBD and anti-EGFP positive control VHH against surface RBD expression cells and surface EGFP expression cells, equal mass mixtures of SR6v7-spacer, SR1-spacer, GP6-spacer, GP3k1k-spacer DNA was used for generating ribosome display pool. For VHH-antigen mapping of VHHs obtained by VHH selection against whole cells, the selection output DNA library from VHH selection against whole cells were mixed with anti-RBD positive control VHH-spacer DNA at mass ratio of 350 ng selection output DNA library to 0.75 ng positive control VHH-spacer DNA, and this DNA mixture was used for generating ribosome display pool.

### VHH-Antigen Mapping: Sequencing and Analysis

Sequencing library was sequenced on illumina Miseq using MiSeq Reagent Micro Kit v2 (300-cycles) (illumina). The obtained reads were first separated into VHH reads and antigen DNA barcode reads using their associated 10x Genomics capture sequence. Reads with duplicated Unique Molecular Identifier (UMI) were removed such that only reads with unique UMI were used in the downstream analysis to ensure reads count equals UMI count. The antigen DNA barcode reads were used to assign antigen identity to each compartment (droplet) through the 10× Barcode (which uniquely label each compartment) in the antigen DNA barcode reads. The VHH reads were clustered by their CDR sequence similarity as described previously (see PMID: 34535642), each VHH read was assigned to a compartment through the 10×Barcode in the read, the antigen present in the compartment were assigned to the VHH read. For each VHH cluster, the count of its member sequence over all the antigens included in the experiment were calculated, the count on each antigen is normalized by compartment number by dividing the count by the number of compartments having the antigen. Under the assumption that each compartment includes one cell, this compartment number normalization was referred to as cell number normalized and the compartment normalized count was referred to as count per cell. The count per cell values over all antigens for each VHH cluster was used to calculate the fraction on antigen for the VHH cluster.

SEQUENCES

Lengthy table referenced here
US20250012808A1-20250109-T00001
Please refer to the end of the specification for access instructions.

Lengthy table referenced here
US20250012808A1-20250109-T00002
Please refer to the end of the specification for access instructions.

Lengthy table referenced here
US20250012808A1-20250109-T00003
Please refer to the end of the specification for access instructions.

Lengthy table referenced here
US20250012808A1-20250109-T00004
Please refer to the end of the specification for access instructions.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any clement(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims

### ENUMERATED EMBODIMENTS

The following embodiments are within the scope of the present disclosure. Furthermore, the disclosure encompasses all variations, combinations, and permutations of these embodiments in which one or more limitations, elements, clauses, and descriptive terms from one or more the listed embodiments is introduced into another listed embodiment in this section. For example, any listed embodiment that is dependent on another embodiment can be modified to include one or more limitations found in any other listed embodiment in this section that is dependent on the same base embodiment. Where elements are presented as lists, e.g., in Markush group format, each subgroup of the elements is also disclosed, and any element(s) can be removed from the group. It should be understood that, in general, where the disclosure, or aspects of the disclosure, is/are referred to as comprising particular elements and/or features, certain embodiments of the invention or aspects of the invention consist, or consist essentially of, such elements and/or features. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

1. A method for obtaining a plurality of cognate antibodies or cognate antigen binding fragments that bind to their cognate binding partners present on a plurality of target cells comprising:
(a) incubating a plurality of antibodies or antigen binding fragments with a plurality of target cells comprising one or more binding partners under conditions that allow for binding to the binding partners;
(b) collecting one or more antibodies or antigen binding fragments that bound to their respective cognate binding partner;
   wherein the plurality of cognate antibodies or cognate antigen binding fragments that bind to their respective cognate binding partners are obtained.

2. The method of embodiment 1, wherein the plurality of antibodies or antigen binding fragments is obtained by
(aa) incubating a preliminary plurality of antibodies or antigen binding fragments with a non-target cell population under conditions that allow for binding of one or more antibodies or antigen binding fragments to the non-target cell population;
(bb) collecting one or more antibodies or antigen binding fragments that did not bind to the non-target cell population in (aa),
   wherein the plurality of antibodies or antigen binding fragments is obtained.

3. The method of embodiment 2, wherein the non-target cells do not express, or express lower levels as compared to the target cells of, cognate binding partners.

4. The method of any one of embodiments 1-3, wherein the target cells are live single cells in suspension.

5. The method of any one of embodiments 1-3, wherein the target cells are fixed using fixation reagent such as formaldehyde.

6. The method of any one of embodiments 1-5, wherein the target cells are one or more of a T cell population, a kidney cell population or a bone marrow cell population.

7. The method of any one of embodiments 1-5, wherein the target cells are blood cells.

8. The method of any one of embodiments 1-5, wherein the target cells are cancer cells.

9. The method of any one of embodiments 1-5, or 8, wherein the target cells are leukemia cells or neuroblastoma cells.

10. The method of any one of embodiments 1-5, 8, or 9, wherein the target cells are leukemic T-cell lymphoblasts.

11. The method of any one of embodiments 1-5, wherein the target cells are Jurkat cells, HEK-293 cells, SH-SY5Y cells, or CHO cells.

12. The method of any one of embodiments 1-11, wherein the target cells are cultured for 1 day to 14 days.

13. The method of embodiment 2 or 3, wherein the non-target cells are phylogenetically distant from the target cells.

14. The method of either embodiment 2 or 3, wherein the non-target cells and the target cells are from different organs.

15. The method of any one of embodiments 1-14, wherein collecting the one or more antibodies or antigen binding fragments comprises the steps of (1) incubating the cells in a buffer comprising chelating agents, and (2) retaining the supernatant after fractionation.

16. The method of embodiment 15, wherein the chelating agent is EDTA or EGTA.

17. The method of any one of embodiments 1-16, wherein the target cell expresses one or more cancer driver genes and/or genes carrying cancer driver mutations, and the cognate binding partners are induced by the cancer driver genes and/or genes carrying cancer driver mutations.

18. A method for the identification of one or more cognate antibodies or cognate antigen binding proteins as matching to one or more cognate binding partners, the method comprising:
(a) providing a population of cells that express cognate binding partners;
(b) disrupting and/or decreasing the expression of one or more cognate binding partners in the cells of the population; and
(c) incubating the cell population in (b) with the plurality of cognate antibodies or cognate antigen binding fragments obtained in any one of embodiments 1-17 under conditions that allow for binding of cognate antibodies or cognate antigen binding fragments to their respective cognate binding partners;
   wherein the one or more cognate antibodies or cognate antigen binding fragments is matched to their respective cognate binding partner if their binding to the cell in which the cognate binding partner is disrupted and/or decreased as compared to the binding to the non-disrupted target cells.

19. The method of 18, wherein the expression of the respective cognate binding partner is disrupted using clustered regularly interspaced short palindromic repeats (CRISPR).

20. The method of embodiment 19, wherein the cell also expresses a gene editing nuclease.

21. The method of 20, wherein the gene editing nuclease is an endonuclease.

22. The method of embodiment 20, wherein the gene editing endonuclease is Cas9.

23. The method of any one of embodiments 18-22, further comprising contacting the cell with a ribonucleic acid that is a single-guide RNA (sgRNA).

24. The method of embodiment 18, further comprising contacting the cell with a ribonucleic acid which is either a small interfering RNA (siRNA) or a short hairpin RNA (shRNA), thereby knocking-down the expression of the respective cognate binding partner in the cell.

25. The method of either embodiment 23 or 24, wherein the cognate antibodies or cognate antigen binding fragments are linked to nucleic acids containing their respective coding sequences.

26. The method of embodiment 25, wherein the ribonucleic acid and the nucleic acid encoding the cognate antibody or cognate antibody binding fragment are sequenced by single cell sequencing.

27. The method of any one of embodiments 23-26, wherein the ribonucleic acid sequences are used to identify the cognate binding partner with disrupted expression in the cell.

28. The method of any one of embodiments 23-27, wherein the cell is contacted with plurality of sgRNAs or shRNAs at a multiplicity of infection (MOI) of 0.2, 0.3, 0.4, 0.5, 0.6, 0.7, 0.8 or 0.9.

29. The method of embodiment 28, wherein the plurality of sgRNAs or shRNAs is introduced into the cell through lentiviral transduction.

30. The method of embodiment 28, wherein the plurality of sgRNAs or shRNAs is introduced into the cell through transformation, transfection, or electroporation.

31. The method of embodiment 28, wherein the plurality of sgRNAs or shRNAs comprises 100 to 100,000 different ribonucleic acids encoding different sgRNAs or shRNAs that target expression of different genes encoding different target proteins or target protein domains.

32. A method for the identification of a plurality of cognate antibodies or cognate antigen binding fragments as matching to one or more cognate binding partners, the method comprising:
(a) introducing into a population of cells a plurality of nucleic acids encoding a plurality of binding partners, which result in expression of a plurality of binding partners on the surface of the cells;
(b) incubating the cells obtained in (a) with the plurality of cognate antibodies or cognate antigen binding fragments obtained in any one of embodiments 1-17 under conditions that allow for binding to the plurality of cognate antibodies or cognate antigen binding fragments; and
   wherein the one or more cognate antibodies or cognate antigen binding fragments is matched to their respective cognate binding partner if their binding to the cell in which the cognate binding partner is introduced is increased as compared to the binding to the target cells not introduced with the cognate binding partner.

33. The method of embodiment 32, wherein the cognate binding partner is determined by single-cell sequencing of nucleic acids encoding cognate binding partners and cognate antibodies or cognate antigen binding fragments.

34. The method of any one of embodiments 1-33, wherein the cognate antibody or cognate antigen binding fragments are clustered computationally.

35. The method of embodiment 34, wherein a cluster size of a cognate antibody or cognate antigen binding fragment cluster is assessed by counting the number of sequences in the cluster.

36. The method of embodiment 35, wherein increased or decreased binding of the cognate antibody or cognate antigen binding fragment is determined by an increased or decreased cluster size of the cluster representing the cognate antibody or cognate antigen binding fragment.

37. The method of embodiment 36, wherein the cluster is normalized relative to the total sequence number to obtain a relative cluster size.

38. The method of any one of embodiments 1-37, wherein the antibodies or antigen binding fragments are linked to nucleic acids containing their respective coding sequences.

39. The method of embodiment 38, wherein linking of the antibodies or antigen binding fragments is achieved by ribosome display complexes comprising antibodies or antigen binding fragments, nucleic acids containing coding sequences for the antibodies or antigen binding fragments, and ribosomes.

40. The method of embodiment 39, wherein the nucleic acids containing coding sequence are RNA.

41. The method of either embodiment 39 or 40, wherein the ribosomes are from eukaryotic cells.

42. The method of either embodiment 39 or 40, wherein the ribosomes are from prokaryotic cells.

43. The method of any one of embodiments 1-42, wherein the cognate binding partners are proteins.

44. The method of any one of embodiment 1-43, wherein the cognate binding partner proteins are natively expressed proteins.

45. The method of any one of embodiment 1-44, wherein the cognate binding partner proteins are expressed by the cells by introducing nucleic acids containing the coding nucleic acid sequences for the proteins into the cells.

46. The method of embodiment 43, wherein the proteins have post-translational modifications.

47. The method of either embodiment 44 or 46, wherein the protein is part of a protein complex.

48. The method of any one of embodiments 1-47, wherein the respective cognate binding partners comprise a lipid group or a sugar group.

49. The method of any one of embodiments 1-42, wherein the respective cognate binding partner is not a protein.

50. The method of any one of embodiments 1-42, wherein the respective cognate binding partner is a lipid or a sugar.

51. The method of any one of embodiments 1-50, wherein the respective cognate binding partner is from a mammal.

52. The method of any one of embodiments 1-51, wherein the respective cognate binding partner is from a mouse or a human.

53. The method of any one of embodiments 1-48, 51, or 52, wherein the respective cognate binding partner is a cell surface protein.

54. The method of any one of embodiments 1-48, 51, or 52, wherein the respective cognate binding partner is a membrane protein or membrane protein domain, an intracellular protein, a cytosolic protein, or a nuclear protein.

55. The method of any one of embodiments 1-42, wherein the respective cognate binding partner is a peptide.

56. The method of embodiment 55, wherein the peptide is 2 amino acids to 15 amino acids in length.

57. The method of either embodiment 55 or 56, wherein the peptide is part of a peptide-carrier protein fusion.

58. The method of any one of embodiment 55-57, wherein the peptide is part of a peptide-major histocompatibility complex (MHC) class I complex.

59. The method of any one of embodiments 1-48, 51, or 52, wherein the respective cognate binding partner is a protein that mediates cell-to-cell interactions.

60. The method of any one of embodiments 1-48, 51 or 52, wherein the respective cognate binding partner is a protein that is overexpressed in in cancer cells or a tumor.

61. The method of any one of embodiments 1-48, 51 or 52, wherein the respective cognate binding partner is an ecto −nucleoside triphosphate diphosphohydrolase 1, E-NTPDasel (CD39), a T cell immunoglobulin and mucin domain-3 (Tim3), a cluster of differentiation 28 (CD28), a cluster of differentiation 4 (CD4), or a stimulator of interferon gene (STING).

62. The method of any one of embodiments 1-61, wherein the cognate antibody or cognate antigen binding fragment has an affinity for the respective cognate binding partner of a KD of less than 10 μM.

63. The method of any one of embodiments 1-62, wherein the respective cognate binding partner is a cancer drug target or a cancer immunotherapy target.

64. A method for the detection of cognate binding partners in a sample comprising:
(a) incubating a sample with a plurality of ribosome display complexes, wherein the ribosome display complexes comprise antibodies or antigen binding fragments, nucleic acids containing coding sequences for the antibodies or antigen binding fragments, and ribosomes, under conditions that allow for binding of the plurality of cognate antibodies or cognate antigen binding fragments to the sample;
(b) detecting the ribosome display complexes that bound to the sample; and
(c) identifying cognate antibodies or cognate antigen binding fragments that bound to the sample, thereby detecting the cognate binding partners in the sample.

65. The method of embodiment 64, wherein at least one nucleic acid contains a unique barcode in the open reading frame (ORF).

66. The method of any one of embodiments 1-65, wherein the cognate antibodies or cognate antigen binding fragments are cognate nanobodies.

67. The method of any one of embodiments 1-65, wherein the cognate antibody or cognate antigen binding fragment is a variable domain of the heavy chain (VHH).

68. The method of any one of embodiments 1-65, wherein the cognate antibody or cognate antigen binding fragment is modified to alter stability, in vivo half-life, neutralizing activity and/or dimerization.

69. The method of any one of embodiments 1-65, wherein the cognate antibody or cognate antigen binding fragment is a fusion protein.

70. The method of any one of embodiments 1-65, wherein the cognate antibody or cognate antigen binding fragment is fused to another antibody or antibody fragment, Fc domain, antigen binding domain, glutathione S-transferase (GST), and/or serum albumin.

71. The method of any one of embodiments 1-70, wherein the respective cognate binding partner is an antigen.

72. The method of embodiment 64, wherein the sample is a population selected from the group of single cells, tissue slices, organs, or organisms.

73. The method of embodiment 65, wherein step (b) and/or step (c) is achieved by sequencing the barcodes.

74. The method of any of embodiments 65-73, wherein the barcodes are the nucleic acid sequence of CDR1 or CDR2 or CDR3 of the cognate antibodies or cognate antigen binding fragments.

75. The method of embodiment 64, wherein step (b) is achieved by using secondary antibodies.

76. The method of embodiment 64, wherein step (b) is achieved by fluorescence in situ hybridization.

77. The method of any of embodiments 1-76, wherein a plurality of nucleic acids encoding the cognate antibodies or cognate antigen binding fragments is used to determine a surface proteome of a cell in the target cell population.

78. The method of any of embodiments 1-76, wherein a surface proteome of a cell in the target cell population is determined using flow cytometry, imaging, proteomic analysis or functional analysis.

79. The method of any of embodiments 1-78, further comprising synthesizing one or more sequences from one or more clusters of cognate antibodies or antigen binding fragments for cloning of the respective genes and testing the respective proteins.

80. The method of any one of embodiments 1-79, wherein the cognate antibodies or cognate antigen binding fragments are allowed to bind to their respective cognate binding partners in the presence of ribonuclease inhibitors.

81. The method of embodiment 80, wherein the ribonuclease inhibitor is a protein or a chemical.

82. The method of embodiment 80, wherein the ribonuclease inhibitor is Ribonucleoside Vanadyl Complex.

83. A composition comprising a cognate antibody or cognate antigen binding fragment associated with a respective cognate binding partner, a target cell and a nucleic acid inside the target cell encoding the respective cognate binding partner.

84. The composition of embodiment 83, wherein the target cell is a T cell, a kidney cell or a bone marrow cell.

85. The composition of embodiment 83, wherein the target cell is a blood cell.

86. The composition of embodiment 83, wherein the target cell is a cancer cell.

87. The composition of embodiment 83, wherein the target cell is a leukemia cell or a neuroblastoma cell.

88. The composition of embodiment 83, wherein the target cell is a leukemic T-cell lymphoblast.

89. The composition of embodiment 83, wherein the target cell is a Jurkat cell, a HEK-293, an SH-SY5Y cell, or a CHO cell.

90. The composition of any one of embodiments 83-89, wherein the respective cognate binding partner is a cancer drug target.

91. The composition of any one of embodiments 83-90, wherein the respective cognate binding partner is a protein or a protein domain.

92. The composition of embodiment 91, wherein the protein or protein domain has a post-translational modification.

93. The composition of any one of embodiments 83-92, wherein the respective cognate binding partner is part of a protein complex.

94. The composition of any one of embodiments 83-90, wherein the respective cognate binding partner comprises a lipid group or a sugar group.

95. The composition of any one of embodiments 83-90, wherein the respective cognate binding partner is not a protein.

96. The composition of any one of embodiments 83-90, wherein the respective cognate binding partner is a lipid.

97. The composition of any one of embodiments 83-90, wherein the respective cognate binding partner is a sugar.

98. The composition of any one of embodiments 83-97, wherein the respective cognate binding partner is from a mammal.

99. The composition of any one of embodiments 83-98, wherein the respective cognate binding partner is from a mouse.

100. The composition of any one of embodiments 83-98, wherein the respective cognate binding partner is from a human.

101. The composition of any one of embodiments 83-93, 98, or 99, wherein the respective cognate binding partner is a cell surface protein.

102. The composition of any one of embodiments 83-93, 98, or 99, wherein the respective cognate binding partner is a membrane protein or membrane protein domain.

103. The composition of any one of embodiments 83-93, 98, or 99, wherein the respective cognate binding partner is an intracellular protein.

104. The composition of any one of embodiments 83-93, 98, or 99, wherein the respective cognate binding partner is a cytosolic protein.

105. The composition of any one of embodiments 83-93, 98, or 99, wherein the respective cognate binding partner is a nuclear protein.

106. The composition of any one of embodiments 83-93, 98, or 99. wherein the respective cognate binding partner is a peptide.

LENGTHY TABLES
The patent application contains a lengthy table section. A copy of the table is available in electronic form from the USPTO web site (https://seqdata.uspto.gov/?pageRequest=docDetail&DocID=US20250012808A1). An electronic copy of the table will also be available from the USPTO upon request and payment of the fee set forth in 37 CFR 1.19(b)(3).

1. A method for the identification of respective binding partners for a plurality of unique cognate antibodies or antigen binding fragments thereof, the method comprising:
(a) introducing into a population of cells a plurality of nucleic acids encoding a plurality of binding partners, which results in expression of a plurality of unique binding partners on the surface of the cells; and
(b) incubating the cells obtained in step (a) with a plurality of unique antibodies or antigen binding fragments thereof under conditions that allow for binding to the plurality of unique binding partners;
wherein the plurality of unique cognate antibodies or antigen binding fragments thereof is matched to their respective cognate binding partner if their binding to a cell in which the cognate binding partner is introduced is higher as compared to the binding to a cell in which the nucleic acid encoding the cognate binding partner was not expressed or expressed at a lower level.

2-4. (canceled)

5. A method for the identification of respective binding partners for a plurality of unique cognate antibodies or antigen binding fragments thereof, the method comprising:
(a) providing a population of cells that express one or more cognate binding partners;
(b) disrupting and/or decreasing the expression of one or more cognate binding partners in one or more cells of the population;
(c) incubating the population of cells in step (b) with a plurality of unique antibodies or antigen binding fragments thereof under conditions that allow for the binding of cognate antibodies or cognate antigen binding fragments thereof to their respective cognate binding partner; and
(d) determining which cognate antibodies or cognate antigen binding fragments thereof did not bind or had decreased binding to one or more cells of the population with disrupted and/or decreased cognate binding partner expression;
wherein the respective cognate binding partners for the plurality of cognate antibodies or cognate antigen binding fragments thereof is identified when the cognate antibodies or cognate antigen binding fragments thereof is determined to have not bound or had lower binding to the cell in which the cognate binding partner is disrupted and/or decreased.

6-37. (canceled)

38. A method for obtaining a plurality of unique cognate antibodies or antigen binding fragments thereof that bind to their respective cognate binding partner present on a surface comprising:
(a) incubating a plurality of unique antibodies or antigen binding fragments thereof with one or more binding partners bound to the surface under conditions that allow for binding of the antibodies or antigen binding fragments thereof to their respective cognate binding partner; and
(b) collecting a plurality of antibodies or antigen binding fragments thereof that had bound to their respective cognate binding partner;
wherein the plurality of unique cognate antibodies or antigen binding fragments thereof that bind to their respective cognate binding partner are obtained.

39-78. (canceled)

79. A method for the detection of respective cognate binding partners in a sample comprising:
(a) incubating a sample comprising a population of cells expressing cognate binding partners with a plurality of ribosome display complexes, wherein the ribosome display complexes comprise:
(1) a plurality of unique antibodies or antigen binding fragments thereof,
(2) nucleic acids encoding the plurality of unique antibodies or antigen binding fragments thereof, and
(3) ribosomes,
wherein the sample and the plurality of ribosome display complexes are incubated under conditions that allow for binding of the plurality of the antibodies or antigen binding fragments thereof to their respective cognate binding partners expressed by the cells of the sample;
(b) detecting the ribosome display complexes that bound to their cognate binding partners in the sample; and
(c) quantifying the cognate antibodies or cognate antigen binding fragments thereof bound to their cognate binding partners expressed by the cells of the sample, thereby detecting the respective cognate binding partners in the sample.

80. The method of claim 79, wherein at least one nucleic acid encoding an antibody or antigen binding fragment thereof comprises a unique barcode in the open reading frame (ORF).

81. The method of claim 79, wherein the sample is selected from the group consisting of single cells, tissue slices, organs, and organisms.

82. The method of claim 80, wherein step (c) further comprises sequencing one or more of the barcodes.

83. The method of claim 80, wherein the barcodes are the nucleic acid sequence of CDR1, CDR2, or CDR3 of the cognate antibodies or antigen binding fragments thereof.

84. The method of claim 79, wherein step (b) is achieved by using secondary antibodies or by fluorescence in situ hybridization.

85. (canceled)

86. The method of claim 79, wherein:
a plurality of nucleic acids encoding the antibodies or antigen binding fragments thereof is used to determine a surface proteome of a cell.

87. (canceled)

88. The method of either claim 86, wherein the surface proteome of a cell in the population of cells is determined using flow cytometry, imaging, proteomic analysis, or functional analysis.

89. The method of claim 88 further comprising sequencing a plurality of nucleic acids encoding the antibodies or antigen binding fragments thereof, clustering the nucleic acid sequences computationally, synthesizing one or more sequences from one or more clusters of antibodies or antigen binding fragments thereof for cloning of the respective genes, and testing the respective antibodies or antigen binding fragments thereof.

90. The method of claim 79, wherein the cognate antibodies or cognate antigen binding fragments thereof are allowed to bind to their respective cognate binding partners in the presence of ribonuclease inhibitors.

91-92. (Canceled)

93. The method of claim 79, wherein the antibodies or antigen binding fragments thereof are nanobodies.

94. The method of any claim 79, wherein the antibodies or antigen binding fragments thereof are a variable domain of the heavy chain (VHH).

95. The method of claim 79, wherein the antibodies or antigen binding fragments thereof are modified to alter stability, aggregation propensity, in vivo half-life, neutralizing activity, and/or dimerization.

96. The method of claim 79, wherein the antibodies or antigen binding fragments thereof are fusion proteins.

97. The method of claim 79, wherein the antibodies or antigen binding fragments thereof are fused to another antibody or antibody fragment thereof, Fc domain, antigen binding domain, glutathione S-transferase (GST), and/or serum albumin; and/or modified with a small molecule.

98. The method of claim 79, wherein the respective cognate binding partner is an antigen.

99. A composition comprising a cognate antibody or cognate antigen binding fragment thereof associated with a respective cognate binding partner, a target cell, and a nucleic acid inside the target cell encoding, or interfering with the expression of, the respective cognate binding partner.

100-124. (canceled)
